SEQUENCE LISTING (14 sequences)

SEQ ID NO: 1 - protein, 687 aa, Homo sapiens
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 2 - protein, 317 aa, Homo sapiens [sequence omitted]

SEQ ID NO: 3 - protein, 384 aa, Homo sapiens
MARALVQLWAICMLRVALATVYFQEEFLDGEHWRNRWLQSTNDSRFGHFRLSSGKFYGHKEKDKGLQTTQNGRFYAISARFKPFSNKGKTLVIQYTVKHEQKMDCGGGYIKVFPADIDQKNLNGKSQYYIMFGPDICGFDIKKVHVILHFKNKYHENKKLIRCKVDGFTHLYTLILRPDLSYDVKIDGQSIESGSIEYDWNLTSLKKETSPAESKDWEQTKDNKAQDWEKHFLDASTSKQSDWNGDLDGDWPAPMLQKPPYQDGLKPEGIHKDVWLHRKMKNTDYLTQYDLSEFENIGAIGLELWQVRSGTIFDNFLITDDEEYADNFGKATWGETKGPEREMDAIQAKEEMKKAREEEEEELLSGKINRHEHYFNQFHRRNEL

SEQ ID NO: 4 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 5 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 6 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 7 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 8 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 9 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 10 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 11 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 12 - protein, 1134 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKPGIPAAEVGIGVVAEADAADAAGFPVPPDMEDDYEPELLLMPSNQPVNQPILAAAQSLHREATKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 13 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 14 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

(19) United States
(12) Patent Application Publication
(10) Pub. No.: US 2023/0355669 A1
(43) Pub. Date: Nov. 9, 2023

(54) SYSTEMS AND METHODS FOR PROMOTING REMYELINATION WITH FACTORS DERIVED FROM UMBILICAL CORD BLOOD MACROPHAGES

(71) Applicant: Duke University, Durham, NC (US)

(72) Inventors: Anthony Filiano, Durham, NC (US); Li Xu, Durham, NC (US); Joanne Kurtzberg, Durham, NC (US)

(73) Assignee: Duke University, Durham, NC (US)

(21) Appl. No.: 18/312,719

(22) Filed: May 5, 2023

Related U.S. Application Data
(60) Provisional application No. 63/338,668, filed on May 5, 2022.

(51) Int. Cl. A61K 35/15; C12N 5/0786; A61P 25/28

(52) U.S. Cl. CPC A61K 35/15; C12N 5/0645; A61P 25/28; C12N 2500/25; C12N 2501/135; C12N 2501/165; C12N 2501/73

(57) ABSTRACT

Compositions and methods of promoting myelination in neurons are disclosed. The compositions include factors present in an umbilical cord blood derived macrophage cell population culture medium which can promote maturation of oligodendrocytes to promote myelination of neurons. Clinal and non-clinical uses of the compositions are also disclosed.

### CROSS-REFERENCE TO RELATED APPLICATIONS

This application claims priority from Provisional Application No. 63/338,668, filed May 5, 2022, the entire contents of which is hereby incorporated by reference for all purposes.

### SEQUENCE LISTING

This application contains a Sequence Listing that has been submitted in WIPO ST.26 .xml format via EFS-Web and is hereby incorporated by reference in its entirety. The .xml copy is named 23-2081-US_Sequence_Listing.xml, and is 24 kilobytes in size.

### FIELD

The present disclosure provides compositions and methods to promote myelination in neurons. The present disclosure further provides methods for using the compositions provided herein for clinical and non-clinical purposes.

### BACKGROUND

A demyelinating disorder is a condition that reduces or causes damage to the protective covering (myelin sheath) that surrounds certain neurons in the brain, for example nerves leading to the eye and spinal cord. Myelin sheaths act to insulate neurons and increase the rate at which electrical impulses pass along the axons. This function is essential for normal motor, sensory and cognitive function. In the central nervous system, myelin is formed by glial cells called oligodendrocytes. When the myelin sheath is damaged, nerve impulses slow or even stop, causing neurological problems. Multiple brain diseases including various neurodegenerative autoimmune diseases, leukodystrophies, and peripheral neuropathies can be attributed to demyelination.

Multiple sclerosis (MS) is the most commonly known demyelinating disease of the central nervous system and is a leading cause of chronic neurological disability in young to middle-aged adults, affecting more than 2.5 million people worldwide. In this disorder, the immune system attacks the myelin sheath and causes communication problems between the brain and the rest of the body. Eventually, the disease can cause permanent damage or deterioration of the nerves. In 85% of patients, there is an initial period of episodic neurological dysfunction followed by partial or complete recovery (relapsing-remitting MS, RRMS). Over time, the clinical picture often develops to one of progressive disability (secondary progressive MS, SPMS), while in 15% the illness is progressive from the outset (primary progressive MS, PPMS).

Current therapies are either directed at managing symptoms or inhibiting inflammation with the goal of slowing demyelination and disease progression. Disease-modifying therapies (DMTs) are designed to reduce the frequency and severity of MS attacks. DMTs can also slow the progression of disability and the loss of brain volume mass. The majority of DMTs approved by the FDA are effective at helping to manage RRMS, which affects between 85% and 90% of people diagnosed with this disease. While the 16 current DMTs are licensed for RRMS, only one is approved for PPMS.

However, there is no cure for MS. Most therapeutics are systemic immunosuppressive or immunomodulatory drugs, but these drugs are unable to halt or reverse the disease and have the potential to cause serious adverse events, and ultimately, patients succumb to disease.

There is thus an unmet need for MS treatments that delay, prevent or reverse progression. One of the most tractable strategies to achieve this is to therapeutically enhance endogenous remyelination. Doing so restores nerve conduction and prevents neurodegeneration. There are currently no FDA approved therapies that promote remyelination.

### BRIEF SUMMARY

These and other novel features and advantages of the disclosure will be fully understood from the following detailed description and the accompanying drawings.

In some aspects, the current disclosure encompasses a composition comprising at least one factor present in an umbilical cord blood derived macrophage cell population culture medium or a biologically active variant, derivative or fragment thereof, and at least one excipient, wherein the composition promotes myelination of a neuron in presence of a primary oligodendrocyte precursor cell (OPCs). In some aspects, the umbilical cord blood derived macrophage cell population comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6 and IL-10. In some aspects, the umbilical cord blood derived macrophage cell population comprises a DUOC-01 cell product. In some aspects, the composition comprises at least two, or at least 5, or at least 10, or at least 50, or at least 100, or at least 150, or at least 200, or at least 300 or more factors present in an umbilical cord blood derived macrophage cell population, or biologically active variants, derivatives, or fragments thereof. In some aspects, the composition comprises the conditioned media as disclosed herein. In some aspects, the composition comprises Remy-Macs. In some aspects, the composition comprises one or more of the factors listed in Table 1.

In some aspects, administering an effective amount of the composition as disclosed herein, in a subject in need thereof can reduce, reverse, or treat a demyelination condition. Non-limiting examples of demyelination conditions include multiple sclerosis, leukodystrophies, spinal cord injury, peripheral nerve damage, Parkinson's disease, amyotrophic lateral sclerosis (ALS), or Alzheimer's disease. In some aspects, the subject is a child suffering from leukodystrophies and undergoing donor umbilical cord blood transplantation after myeloablative conditioning. In some aspects, the subject is suffering from multiple sclerosis. In some aspects, the administering can be done via any route of administration, for example via a subcutaneous, intrathecal, intramuscular, intranasal, parenteral, transepithelial, or epidural route.

In some aspects, the at least one factor can drive the differentiation of the OPC to mature myelin basic protein expressing oligodendrocyte. In some aspects, the OPCs are present in an in vitro or ex vivo culture. In some aspects, the OPCs are present or administered in vivo.

In some aspects, the current disclosure also encompasses a method of promoting myelination of neurons, the method comprising: obtaining at least one factor present in an umbilical cord blood derived macrophage cell population culture medium, or a biologically active variant, derivative or fragment thereof; and contacting at least a primary oligodendrocyte precursor (OPC) cell with the at least one factor, wherein the OPC matures to a myelin basic protein expressing oligodendrocyte, thus promoting myelination is neurons. In some aspects of the method as disclosed herein, the umbilical cord blood derived macrophage cell population comprises cells expressing one or more of CD45, CD11b, CD14, CD16, CD206, CD163, Iba1, HLA-DR, TREM 2, and iNOS macrophage or microglia markers; and wherein such cells secrete IL-6 and IL-10.

In some aspects of the disclosed method, the contacting of at least a primary oligodendrocyte precursor (OPC) cell with the at least one factor, wherein the OPC matures to a myelin basic protein expressing oligodendrocyte, thus promoting myelination in neurons is performed in vitro or ex vivo. In some aspects, the contacting step is performed in vivo by administering to a subject in need thereof the at least one factor present in the umbilical cord blood derived macrophage cell population. In some aspects of the method, the administering is done via a subcutaneous, intrathecal, intramuscular, intranasal, parenteral, transepithelial, or epidural route.

In some aspects, the subject has symptoms of or is diagnosed with a demyelination condition, for example, multiple sclerosis, leukodystrophies, spinal cord injury, peripheral nerve damage, Parkinson's disease, amyotrophic lateral sclerosis (ALS), or Alzheimer's disease.

In some aspects, the current disclosure also encompasses a method of treating a subject in need thereof, the method comprising: determining an amount of at least one factor secreted by an umbilical cord blood derived macrophage cell population, in a biological sample of the subject in need thereof, comparing the amount of the at least one factor with a standard to obtain a differential amount; determining a treatment regimen for the subject, wherein the treatment regimen comprises administering to the subject in need thereof a therapeutically effective amount of a composition comprising the at least one factor, or a biologically active variant, derivative or fragment thereof. In some aspects, the subject is exhibiting symptoms of or is diagnosed with a demyelination condition, for example, multiple sclerosis, leukodystrophies, spinal cord injury, peripheral nerve damage, Parkinson's disease, amyotrophic lateral sclerosis (ALS), or Alzheimer's disease.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is a brightfield microscopic image of the macrophage-like cells manufactured from umbilical cord blood units.

FIG. 1B is a FACS based cell sorting of the cells based on expression of ligands including CD11b, CD45, SSC, TREM2, CD14 and CD3.

FIG. 1C shows t-SNE plots demonstrating 7 different clusters in 3 DUOC cultures. DUOC-01 cells were cultured for 21 days, and single, live cells were sorted by FACS. RNA sequencing libraries were generated on the ddSEq platform with Illumina Bio-Rad's SureCell WTA 3′ Library Prep for Peripheral Blood Mononuclear Cells.

FIG. 1D shows application of a microglial signature on the microarray data from DUOC-01 cultures. Compared to peripheral macrophages, DUOC-01 cells are enriched for the microglial signature.

FIG. 2A is a schematic of organotypic slice culture system.

FIG. 2B are representative photographs of the culture at various stages (days in vitro, DIV).

FIG. 2C shows immunohistochemical staining used to check the presence and morphology of cell types within the mouse fore-brain slices in ex-vivo culture conditions. A representative image showing Astrocytes (GFAP, pink), Neurons (NeuN, red), oligodendrocytes (Olig2, green) and microglial (Iba1, blue) at day 9 in vitro is provided.

FIG. 2D shows immunohistochemical staining to check for myelinated axons in cerebellar slices in ex-vivo culture conditions. A representative image showing neuron (MAP2, red) and myeline sheath (MBP, green) at 9 days in vitro is provided.

FIG. 2E is a schematic of organotypic cerebellar culture set-up, LPC demyelination and DUOC-01 treatment.

FIG. 2F is a representative immunohistological image of culture after LPC and DUOC-01 treatments, myelin basic protein (MBP) is shown in green, neurofilament (NFSMi312) stain is shown in pink. MBP and neurofilament co-localization is shown in white.

FIG. 2G is a graph of the percent co-localization of MBP and NFSMMi312, for LPC, no-LPC control and DUOC-01 treated groups. Significantly higher myelination was observed in the DUOC-01 treated group, compared to LPC treated control (*p<0.00001).

FIG. 3A is a schematic of EAE induction, cell injection and clinical monitoring protocol. The insert shows anatomy of cisterna magna (injection site).

FIG. 3B is a graph showing the average EAE scores of mice injected with Ringer's or DUOC-01 (N=9 mice per group). Data is presented as mean±SEM. Repeated measures Two-way ANOVA for DUOC-01 p<0.05; for interaction and time p<0.001; post-hoc **p<0.01 and *p<0.05.

FIG. 4A is a schematic of OPC culture set-up and cell treatment protocol.

FIG. 4B is a microscopic image showing formation of oligospheres and morphology of OPC.

FIG. 4C shows that DUOC-01 treatment does not affect the OPC proliferation whether the DUOC-01 cells are directly added to the OPC culture or in the transwell (N=12).

FIG. 4D shows that direct DUOC-01 treatment or the treatment of DUOC-01 in a transwell both drive the OPC culture to more mature phenotype. Mature oligodendrocyte markers Myelin Basic Protein (MBP) and Myelin Oligodendrocyte Glycoprotein (MOG), both were upregulated after treatment.

FIG. 4E shows immunohistochemistry with Olig2 and MBP staining for OPC and OPC with cell treatment.

FIG. 4F shows quantitation of the IHC for MBP staining. Significantly higher MBP expression could be seen after direct DUOC-01 treatment or the treatment from DUOC-01 in the transwell. Data is presented as mean±SEM. Repeated measures Two-way ANOVA for DUOC-01 p<0.01.

FIG. 5A is data showing that Remy-Macs promote the maturation of OPCs. Primary mouse OPC cultures were up from oligospheres originally isolated from mouse brain. OPC cultures were either cultured in OPC media (control; CTL) or with Remy-Macs (conditioned media taken from macrophages derived from umbilical cord blood monocytes). Myelin basic protein (MBP) is a marker for mature oligodendrocytes. MBP mRNA was analyzed by quantitative real-time PCR. Remy-Macs increased the expression of MBP compared to CTL (N=27, t-test, ****P<0.0001).

FIG. 5B are whole well tile scans of culture plates with OPCs cultured in Remy-Macs.

FIG. 5C is data showing that Remy-Macs increased the number of MBP expressing cells compared to the control media (N=4, t-test, *P<0.05).

FIG. 5D are representative images of OPC cultures treated with control media or Remy-Macs (conditioned media) and immuno-labeled for MBP. In cultures treated with Remy-Macs, cells displayed complex highly ramified processes demonstrating the maturation of OPC into mature oligodendrocytes. In cultures treated with control media, cells displayed simple morphology demonstrating immature OPCs.

### DETAILED DESCRIPTION

Unless otherwise defined, all technical terms used herein have the same meaning as commonly understood by one of ordinary skill in the art to which this disclosure belongs.

In some aspects, the current disclosure is based on the surprising discovery that the promotion of remyelination induced by macrophages derived from umbilical cord blood (for example, cell product DUOC-01, as disclosed in US application U.S. Ser. No. 16/477,167, the entirety of which is hereby incorporated by reference) can be replicated by using conditioned media. The conditioned media is the result of culturing an umbilical cord blood derived macrophage cell population, like DUOC-01. The processed conditioned media clarified of debris is referred herein as Remy-Macs.

In the current disclosure the inventors have found that conditioned media or clarified Remy-Macs are rich in factors that promote myelination by several mechanisms. In some aspects, the current disclosure encompasses compositions comprising at least one such factor secreted from an umbilical cord blood derived macrophage cell population, like DUOC-01, wherein the composition promotes myelination of a neuron in presence of a primary oligodendrocyte precursor cell (OPCs), by converting OPCs to mature OPC expressing at least higher levels of Myelin Binding Protein (MBP) than untreated primary OPCs.

In view of the present disclosure, the methods and compositions described herein can be configured by the person of ordinary skill in the art to meet the desired need. In general, the disclosed compositions and methods provide improvements in treatment of demyelinating conditions. In some aspects, the therapeutic methods, therapeutic combinations and pharmaceutical compositions provided herein are useful for promoting myelination while reducing demyelination, for slowing progression or reducing frequency of relapse or both of a demyelination condition, and/or enhancing physical ability of a subject having a demyelination condition. The term “promote” or “promoting” myelination as used herein refers to an overall increase in the level of myelin on a neuron or a group of neurons. At the very least, this involves an increase in the synthesis of myelin proteins and lipids by oligodendrocytes through a coordinated expression of genes that encode myelin components. In some aspects, the compositions and methods as disclosed herein can enhance the production of myelin by inducing expression, production, stability or transport of myelin components in oligodendrocytes. In some aspects, the compositions and methods as disclosed herein may also reduce demyelination or damage to existing myelin or myelinated neurons. In some aspects, the term “reducing”, “reduce” and “reduction” as used herein include to reverse, arrest, slow, retard or stabilize demyelination, progression of a demyelination condition, or an effect of such progression. For example, in some aspects, reduction is only partial, such as a slowing or retarding of progression of a demyelination condition. In other aspects, inhibition is more complete, such as an arrest or even reversal of such progression.

### I. Compositions

In some aspects, the current disclosure encompasses a composition comprising at least one factor present in an umbilical cord blood derived macrophage cell population culture medium and at least one excipient or carrier, wherein the composition promotes myelination of a neuron in presence of a primary oligodendrocyte precursor cell (OPCs). The umbilical cord blood derived macrophage cell population of the current disclosure is a population of cultured cord blood derived macrophage cells which are enriched in CD14 and can be further characterized by several other markers. For example, in some aspects, the population of cells is enriched for one or more of CD45, CD11b, CD16, CD206, CD163, Iba1, HLA-DR, TREM 2, and iNOS macrophage or microglia markers. In some aspects, the cells in the population are also characterized by their secretion of IL-6 and IL-10. In some aspects, the umbilical cord blood derived macrophage cell population may correspond to the cell product DUOC-01. During differentiation into such a macrophage cell population or after maturation or both, the condition media becomes rich in factors that support OPC maturation. As used herein, the OPC maturation corresponds with at least an enhancement in the levels of Myelin Binding Protein (MBP) expression in the oligodendrocytes in comparison to primary OPCs. In some aspects, the mature oligodendrocytes show further enhanced expression of myelin proteins and lipids that are involved in the production of myelin sheath.

In some aspects, the current disclosure encompasses a composition comprising at least one such factor secreted from the cell population as described herein into the media. Once the factor is secreted into the media the media is then considered “conditioned media.” The term “conditioned media” as used herein, encompasses the cell secretome, that is the media comprising collection of polypeptides secreted through classical secretion pathways, polypeptides shed from the cell-surface, and intracellular polypeptides released through non-classical secretion pathways that regulate cell-to-cell and cell-to-extracellular matrix interactions. In some aspects, these factors include multiple enzymes, growth factors, cytokines and hormones or other soluble mediators. Though primarily polypeptides, the conditioned media may also comprise other factors, including small molecules, nucleic acids and lipids. With respect to the current disclosure, the conditioned media is the result of culturing an umbilical cord blood derived macrophage cell population, like DUOC-01. As used herein, the term “Remy-Macs” refers to the processed conditioned media clarified of debris obtained from growth of DUOC-01 cell product. Both the conditioned media as provided herein, and the Remy-Macs were found to comprise multiple factors that promote myelination. In some aspects, the at least one factor as disclosed herein is secreted by at least one cell type in the population of umbilical cord blood derived macrophage cells, for example a DUOC-01 cell population as described herein. In some aspects, the factor is produced from more than one cell type in the cell population as disclosed herein. In some aspects, the factor may be a small molecule, an amino acid, a polypeptide, a nucleic acid, a polynucleotide chain, or a lipid. In some aspects, the factor is a polypeptide. In some aspects, the factor is a polypeptide selected from any one of the 357 proteins that were found to be present in the conditioned media derived Remy-Macs as provided in Table 1. In some aspects, the polypeptide is a biologically active variant, derivative or fragment of a polypeptide as disclosed in Table 1. In some aspects, the factor is polypeptide selected from Transglutaminase-2 (TGM2, Uniprot ID: P21980, SEQ ID NO: 1), Apolipoprotein E (APOE, Uniprot ID: P02649, SEQ ID NO: 2), Calreticulin (CALR, Uniprot ID: Q96L12, SEQ ID NO: 3), Collagen Type I Alpha 1 Chain (COL1A1, Uniprot ID: P02452, SEQ ID NO: 4), Alpha-2-Macroglobulin (A2M, Uniprot ID: P01023, SEQ ID NO: 4), Complement C1q B Chain (C1QB, Uniprot ID: P02746, SEQ ID NO: 6), Heat Shock Protein 90 Beta Family Member 1 (HSP90B1, Uniprot ID: P14625, SEQ ID NO: 7), Heat Shock Protein 90 Alpha Family Class A Member 1 (HSP90AA1, Uniprot ID: P07900, SEQ ID NO: 8), Lactotransferrin (LTF, Uniprot ID: P02788, SEQ ID NO: 9), Matrix Metallopeptidase 9 (MMP9, Uniprot ID: P14780, SEQ ID NO: 10), Thrombospondin 1 (THBS1, Uniprot ID: P07996, SEQ ID NO: 11), Vinculin (VCL, Uniprot ID: P18206, SEQ ID NO: 12), Plasminogen (PLG, Uniprot ID: P00747, SEQ ID NO: 13), or Serpin Family C Member 1 (SERPINC1, Uniprot ID: P05155, SEQ ID NO: 14). In some aspects, the polypeptide is a biologically active variant, derivative or fragment of TGM2, APOE, CALR, COL1A1, A2M, CALR, C1QB, HSP90B1, HSP90AA1, LTF, MMP9, THBS1, VCL, PLG, or SERPINC1. In some aspects, the polypeptide is a biologically active variant, derivative or fragment of a sequence corresponding to a sequence as provided in SEQ ID NO: 1-14. In some aspects, the polypeptide sequence is at least about 60%, or at least about 70%, or at least about 80%, or at least about 90%, or at least about 100% identical to as sequence as provided in SEQ ID NO: 1-14.

In some aspects, the current disclosure encompasses a composition comprising at least one, at least 2, at least 3, at least 4, at least 5, at least 10, at least 50, at least 100, at least 150, at least 200, at least 250, at least 300, at least 350 or more factors present in the conditioned media of DUOC-01 cells or biologically active variants, derivatives or fragments thereof. In some aspects, the composition as disclosed herein comprises the entire conditioned medium as disclosed herein. In some aspects, the composition comprises the conditioned media devoid of debris or particulate matter, referred herein as Remy-Macs.

In some aspects, the one or more factors for use in the current disclosure for supporting remyelination, may be isolated from the Remy-Macs or conditioned media used for growth of the DUOC-1 cell product. In some aspects, the one or more factors for use in the current disclosure for supporting myelination, may be part of a composition comprising the Remy-Macs or conditioned media used for growth of the DUOC-1 cell product or an equivalent cell population. In some aspects, the one or more factors or biologically active variants, derivatives, or fragments thereof, for use in the current disclosure may be isolated from a cell that is normally present in the DUOC-01 cell product. In some aspects, the cell is an isolated cell or a cell line. In some aspects, the one or more factors or biologically active variants, derivatives, or fragments thereof, may be isolated from an engineered cell or engineered cell line expressing the factor. In some aspects the engineered cell is a prokaryotic cell. In some aspects, the engineered cell is a eukaryotic cell. In some aspects, the engineered cell is derived from is a OPC cell line, Schwann cell line, stem cell, CHO cell line, or HEK293, PERC6, COS-1, HeLa, VERO, a mouse hybridoma cell line, COS-7 or mouse myeloma cell line. In some aspects, the one or more factors or biologically active variants, derivatives or fragments thereof are produced in vitro using known methods in the art. In some aspects, the one or more factors or biologically active variants, derivatives or fragments thereof are produced in vitro or in vivo or a combination thereof using known methods in the art.

### Pharmaceutical Compositions

In some aspects, the current disclosure also encompasses pharmaceutical compositions comprising at least a factor as disclosed herein and at least a pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science. In each of the aspects described herein, a composition of the disclosure may optionally comprise one or more additional drug or therapeutically active agent in addition to the at least one factor disclosed herein. Thus, in addition to the therapies described herein, one may also provide to the subject other therapies known to be efficacious for treatment of the disease, disorder, or condition.

In some aspects, the excipient may be a diluent. The diluent may be compressible (i.e; plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate

In some aspects, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In some aspects, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In some aspects, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In some aspects, the excipient may be a pH modifier. By way of non-limiting example, the pH modifying agent may be sodium carbonate, sodium bicarbonate, sodium citrate, citric acid, or phosphoric acid.

In some aspects, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. Non-limiting examples of suitable effervescent disintegrants include sodium bicarbonate in combination with citric acid and sodium bicarbonate in combination with tartaric acid.

In some aspects, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, iso-amorphous silicate, and microcrystalline cellulose.

In some aspects, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In some aspects, the excipient may be a lubricant. Non limiting examples of suitable lubricants include minerals such as talc or silica; and fats such as vegetable stearin, magnesium stearate, or stearic acid.

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions described herein can be formulated by any conventional manner using one or more pharmaceutically acceptable carriers or excipients as described in, for example, Remington's Pharmaceutical Sciences (A.R. Gennaro, Ed.), 21^{st} edition, ISBN: 0781746736 (2005), incorporated herein by reference in its entirety. Such formulations will contain a therapeutically effective amount of a biologically active factor described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21^{st} edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The factors of use with the current disclosure can be formulated by known methods for administration to a subject using several routes which include, but are not limited to, parenteral, pulmonary, intrathecal, intradermal, intramuscular, intraperitoneal, intravenous, subcutaneous, intranasal, epidural, ophthalmic, buccal, and rectal. In some aspects, the composition is for administration using a parenteral route. The individual factors may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the factor or reduce dosage frequency or both. Controlled-release preparations can also be used to affect the time of onset of action or other characteristics, such as blood levels of the factor, and consequently affect the occurrence of side effects. Controlled-release preparations may be designed to initially release an amount of a factor(s) that produces the desired therapeutic effect, and gradually and continually release other amounts of the factor to maintain the level of therapeutic effect over an extended period of time. In order to maintain a near-constant level of an factor in the body, the factor can be released from the dosage form at a rate that will replace the amount of factor being metabolized or excreted from the body. The controlled release of an factor may be stimulated by various inducers, e.g., change in pH, change in temperature, enzymes, water, or other physiological conditions or molecules.

### Dosage Forms

The composition can be formulated into various dosage forms and administered by a number of different means that will deliver a therapeutically effective amount of the active ingredient. Such compositions can be administered parenterally, in dosage unit formulations containing conventional nontoxic pharmaceutically acceptable carriers, adjuvants, and vehicles as desired. The term parenteral as used herein includes subcutaneous, intrathecal, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L, Eds., Pharmaceutical Dosage Forms, Marcel Dekker Inc., New York, N.Y. (1980).

For parenteral administration (including cutaneous, subcutaneous, intraocular, intradermal, intravenous, intrathecal, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

In some aspects, a composition comprising the at least one factor as disclosed herein or biologically active variants, derivatives, or fragments thereof, is encapsulated in a suitable vehicle to either aid in the delivery of the composition to target cells, to increase the stability of the composition, or to minimize potential toxicity of the composition. As will be appreciated by a skilled artisan, a variety of vehicles are suitable for delivering a composition of the present disclosure. Non limiting examples of suitable structured fluid delivery systems may include nanoparticles, liposomes, microemulsions, micelles, dendrimers, and other phospholipid-containing systems. Methods of incorporating compositions into delivery vehicles are known in the art.

In some aspects, a liposome delivery vehicle may be utilized. Liposomes, depending upon the aspect, are suitable for delivery of the at least one factor, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition comprising the factor or a biologically active variant, fragment or derivative thereof may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE).

The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9, 12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 Daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the one or more of the factors, may be prepared by any known method of preparing liposomes for drug delivery for example, liposomes may be prepared by sonicating lipids in an aqueous solution, solvent injection, lipid hydration, reverse evaporation, or freeze drying by repeated freezing and thawing. In a preferred aspect the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all the parameters that govern liposome formation may be varied. These parameters include, but are not limited to, temperature, pH, concentration of one or more of a proteotoxicity reducing agent or derivatives thereof, concentration and composition of lipid, concentration of multivalent cations, rate of mixing, presence of and concentration of solvent.

In some aspects, a composition of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one aspect, the structure may be micelles, which are the simplest microemulsion structures that are generally spherical or cylindrical objects. Micelles are like drops of oil in water, and reverse micelles are like drops of water in oil. In an alternative aspect, the microemulsion structure is the lamellae. It comprises consecutive layers of water and oil separated by layers of surfactant. The “oil” of microemulsions optimally comprises phospholipids. Any of the phospholipids detailed above for liposomes are suitable for aspects directed to microemulsions. The one or more of a tricyclic antipsychotics, vasodilator, antibiotic/antiseptic, aryl piperazine or derivatives thereof may be encapsulated in a microemulsion by any method generally known in the art.

In some aspects, the at least one factor or a biologically active variant, derivative, or fragment thereof or combination of factors or their biologically active variants, fragments or derivatives thereof may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome aspects are suitable for use in dendrimer aspects. Any method generally known in the art may be utilized to make dendrimers and to encapsulate compositions of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

Generally, a safe and effective amount of a factor is, for example, that amount that would cause the desired effect in a subject while minimizing undesired side effects. In various aspects, an effective amount of a factor as described herein can substantially induce myelination in a subject in need thereof.

The amount of a composition described herein that can be combined with a pharmaceutically acceptable excipient to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of several individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and aspects of the disclosure, the subject may be a human or any other animal.

In some aspects, there is between about 0.001 mg and about 10 mg of total protein per ml of the composition. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 pg/ml, mg/ml, or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be a polypeptide or combination of polypeptides selected from Table 1 or biologically active variants, fragments or derivatives thereof.

The present disclosure contemplates the administration of a factor to promote myelination.

The present disclosure describes polypeptides, peptides, and proteins for use in various aspects of the present disclosure. For example, specific polypeptides are assayed for their abilities to elicit a remyelination response. In specific aspects, all or part of the proteins of the disclosure can also be synthesized in solution or on a solid support in accordance with conventional techniques. Various automatic synthesizers are commercially available and can be used in accordance with known protocols. See, for example, Stewart and Young, (1984); Tam et al; (1983); Merrifield, (1986); and Barany and Merrifield (1979), each incorporated herein by reference. Alternatively, recombinant DNA technology may be employed wherein a nucleotide sequence which encodes a polypeptide of the disclosure is inserted into an expression vector, transformed or transfected into an appropriate host cell and cultivated under conditions suitable for expression. One aspect of the disclosure includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide described herein may be employed. The generation of recombinant expression vectors, and the elements included therein, are discussed herein. Alternatively, the protein to be produced may be an endogenous protein normally synthesized by the macrophage-like cell population.

Other examples of mammalian host cell lines include, but are not limited to OPC cell line, Schwann cell line, stem cell, CHO cell line, or HEK293, PERC6, COS-1, HeLa, VERO, a mouse hybridoma cell line, COS-7 or mouse myeloma cell line. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

In certain aspects, the present disclosure concerns compositions comprising one or more lipids associated with the factor as disclosed herein. A lipid is a substance that is insoluble in water and extractable with an organic solvent. Compounds other than those specifically described herein are understood by one of skill in the art as lipids and are encompassed by the compositions and methods of the present disclosure. A lipid component and a non-lipid may be attached to one another, either covalently or non-covalently.

A polypeptide/peptide or a nucleic acid encoding it, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid associated composition of the present disclosure is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL)-poxvirus or Superfect (Qiagen)-poxvirus complex is also contemplated.

In certain aspects, a composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or any range there between, of a particular lipid, lipid type, or non-lipid component such as an adjuvant, antigen, peptide, polypeptide, sugar, nucleic acid or other material disclosed herein or as would be known to one of skill in the art. In a non-limiting example, a composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a non-lipid component. Thus, it is contemplated that compositions of the present disclosure may comprise any of the lipids, lipid types or other components in any combination or percentage range.

### II. Methods

In some aspects, the current disclosure encompasses a method of promoting myelination of neurons, the method comprising obtaining at least one factor present in an umbilical cord blood derived macrophage cell population or a biologically active variant, derivative or fragment thereof, and contacting at least a primary oligodendrocyte precursor (OPC) cell with the at least one factor, wherein the OPC matures to a myelin basic protein expressing oligodendrocyte, thus promoting myelination is neurons. In some aspects, the at least one factor may be isolated from the conditioned media or Remy-Macs as disclosed herein. In some aspects, the at least one factor or a biologically active variant, derivative, or fragment thereof, may be expressed and isolated from an engineered cell. In some aspects, the at least one factor or a biologically active variant, derivative, or fragment thereof, may be synthesized in vitro. In some aspects, the at least one factor may be part of a composition comprising Remy-Macs or conditioned media.

In some aspects, the contacting of the at least a primary oligodendrocyte as disclosed in the method herein, may be done in vitro, ex vivo or in vivo depending on the application. For example, in some aspects, the current disclosure encompasses use of the compositions as disclosed herein for laboratory studies requiring maturation of OPCs to mature OPCs capable to enhancing myelination of neurons.

In some aspects, the current disclosure also encompasses use of the composition in vitro or ex vivo for clinical assays or diagnostics to test for myelination factors and efficacy of a treatment of a demyelination condition. Thus, in some aspects, the current disclosure encompasses a method of treating a subject in need thereof, the method comprising: (a) determining an amount of at least one factor secreted by an umbilical cord blood derived macrophage cell population, in a biological sample of the subject in need thereof; (b) comparing the amount of the at least one factor with a standard to obtain a differential amount; (c) determining a treatment regimen for the subject in need thereof, wherein the treatment regimen comprises administering to a subject in need thereof a therapeutically effective amount of a composition comprising the at least one factor, or a biologically active variant, derivative or fragment thereof.

In some aspects, the current disclosure also encompasses method of treatment in a subject in need thereof. In some aspects, the current disclosure encompasses a method of treating a subject in need thereof, the method comprising: (a) administering to the subject in need thereof a therapeutically effective amount of a composition as disclosed herein. In some particular aspects, the current disclosure encompasses a method of treating a subject in need thereof, the method comprising: (a) administering to the subject in need thereof a therapeutically effective amount of a composition as disclosed herein; (b) estimating an amount of at least one macrophage derived factor in a biological sample of the subject after step a; (c) comparing the amount of the at least one factor with a standard to obtain a differential amount; and (d) determining a treatment option for the subject in need thereof based on the differential amount determined in step c.

In some aspects, the biological sample is a bodily fluid. As referred to herein, “bodily-fluid” can include any fluid obtained from a body of a subject, including, but not limited to, blood, cerebrospinal fluid, urine, bile, lymph, saliva, synovial fluid, serous fluid, pleural fluid, amniotic fluid, and the like, or any combination thereof. In some aspects, the biological sample is a cell culture, tissue or an organ sample.

Additionally, the current disclosure also encompasses a method of treatment, the method comprising, administration of a composition comprising the at least one factor as disclosed herein and an excipient, to a subject in need thereof to prevent, treat or reduce the symptoms of a demyelination condition.

In some aspects, the compounds disclosed herein may be administered to the subject by a variety of routes. For example, one or more of the compositions disclosed herein may be administered parenterally (i.e., intrathecally, subcutaneously, intradermally, intravenously, and/or intramuscularly, intracranially, or intraperitoneally). In one aspect, the compounds may be administered in saline or with a pharmaceutically acceptable excipient as described above.

Suitable subjects may include, without limit, humans, as well as companion animals such as cats, dogs, rodents, and horses; research animals such as rabbits, sheep, pigs, dogs, primates, mice, rats, and other rodents; agricultural animals such as cows, cattle, pigs, goats, sheep, horses, deer, chickens, and other fowl; zoo animals; and primates such as chimpanzees, monkeys, and gorillas. The subject can be of any age without limitation. In an aspect, the subject may be a human. Though described herein with respect to human symptoms and conditions, the compositions disclosed herein can be used to treat demyelination conditions in any subject.

The term “demyelination condition” herein refers to a disease, disorder or syndrome in which at least one demyelinating event has occurred. A “demyelinating event” can be a directly observed demyelination lesion or a lesion inferred from a sign or symptom including, but not limited to, optic neuritis, numbness or tingling in a limb, difficulty with speech, loss of balance or coordination, or other motor or sensory problems. In certain aspects, the demyelination condition is associated with an autoimmune response. Examples of demyelination conditions include, but are not limited to, multiple sclerosis and variants thereof, transverse myelitis, encephalomyelitis, Guillain-Barre syndrome, progressive multifocal leukoencephalopathy, leukodystrophies, spinal cord injury, peripheral nerve damage, Parkinson's disease, amyotrophic lateral sclerosis (ALS), or Alzheimer's disease. Variants of multiple sclerosis include, but are not limited to, optic-spinal multiple sclerosis, neuromyelitis optica, acute disseminated encephalomyelitis, Balo concentric sclerosis, Schilder disease and Marburg multiple sclerosis. In a particular aspect, the demyelination condition comprises multiple sclerosis or a variant thereof. In a further particular aspect, the demyelination condition is selected from multiple sclerosis and variants thereof, transverse myelitis, and progressive multifocal leukoencephalopathy. In particular aspects, the demyelination condition is a leukodystrophy, for example childhood leukodystrophy.

The demyelination condition treated by a composition as disclosed herein may be, but is not necessarily, clinically diagnosed. For example, in one aspect, the composition may be administered after the subject is clinically diagnosed with a demyelination condition such as multiple sclerosis. In an alternative aspect, the subject has experienced at least one demyelinating event, but a demyelination condition has not yet been clinically diagnosed. In this aspect of the method, a composition as disclosed herein may be administered before the subject is clinically diagnosed with a demyelination condition such as multiple sclerosis. In one aspect, when the subject has experienced at least one demyelinating event but is not yet clinically diagnosed with a demyelination condition, administering a composition as disclosed herein may delay clinical onset of the demyelination condition. In a particular aspect, clinical onset of multiple sclerosis is delayed. The term “clinical onset” refers to a demyelinating event that confirms diagnosis of the demyelination condition. For example, in the case of multiple sclerosis, clinical onset is at least a second demyelinating event which occurs at least 30 days after a first demyelinating event.

Multiple sclerosis (MS) and other demyelination conditions can result in both neurological (including psychological) and physical effects. Physical effects may induce or result in disability. Initial attacks, i.e., acute outward manifestations of the condition, are often transient, mild or substantially asymptomatic, and are often self-limited. Later attacks, or “relapse”, are often more severe and may be punctuated by periods of remission. Severity and frequency of attacks can be used to classify MS and/or variants thereof into several subtypes: (a) relapse-remitting MS, characterized by unpredictable attacks which may or may not leave permanent neurological deficit and/or disability, followed by periods of remission; (b) primary progressive MS, characterized by a steady decline without attacks; (c) secondary progressive MS, characterized by an initial relapse-remitting period followed by decline without periods of remission; and (d) progressive relapsing MS, characterized by a steady decline since onset with superimposed attacks.

In another aspect a method for inhibiting progression and/or reducing frequency of relapse of a demyelination condition in a subject in need thereof is provided, according to a method of treatment of the disclosure. For example, in one aspect disability progression of MS or a variant thereof may be inhibited. Disability progression refers to physical disability which may or may not be accompanied by neurological symptoms. Examples of such physical disability include, but are not limited to, muscle weakness, abnormal muscle spasms, difficulty in moving such as ambulatory impairment, difficulties with coordination or balance, fatigue, and bladder or bowel difficulties. Disability progression may be quantified on a scale such as the Kurtzke expanded disability status scale (EDSS). The EDSS quantifies disability in eight functional systems (FS's) and allows neurologists to assign a functional system score (FSS) in each. Results on the EDSS are recorded as steps 1 to 10. EDSS steps 1.0 to 4.5 refer to people with multiple sclerosis who are fully ambulatory. EDSS steps 5.0 to 9.5 are defined by impairment of ambulation.

Therefore, in one aspect, treatment according to a method of the disclosure inhibits disability progression in a subject with MS or a variant thereof as measured on the EDSS or equivalent scale.

In another aspect, progression of a neurological and/or psychological effect of the demyelination condition may be inhibited by treatment according to a method of the disclosure. As noted above, MS can have many neurological and/or psychological effects. Examples of such neurological and/or psychological effects, the progression of which may be inhibited, include, but are not limited to, depression, mood swings, emotional lability, euphoria, bipolar syndrome, anxiety, psychosis, cognitive impairments such as short-term and long-term memory problems, forgetfulness, slow word recall, aphasia and dysphasia (impairments to speech comprehension and production), neuropathic pain and dyskinesia.

In yet another aspect of the disclosure, a method is provided for enhancing physical ability of a human subject having a demyelination condition. Enhancing physical ability refers generally to increasing a subject's capacity for movement, such as by increasing muscle strength, tone and/or energy. Examples of physical ability which may be enhanced by the present disclosure include, but are not limited to, a subject's ability to walk (ambulatory movement), coordination and balance, or a subject's use of an arm and/or facial muscles. In a particular aspect, a subject's physical ability is enhanced such that the subject is more ambulatory as measured by the EDSS or equivalent scale.

In certain aspects, a composition comprising the conditioned media or Remy-Macs as disclosed herein may be administered to a subject parenterally at a concentration ranging from about 0.05 mg/kg to about 20 mg/kg. In some aspects, the composition disclosed herein may be administered to a subject intravenously at a concentration of about 0.05 mg/kg, about 0.1 mg/kg, about 0.5 mg/kg, about 1 mg/kg, about 2 mg/kg, about 3 mg/kg, about 4 mg/kg, about 5 mg/kg, about 10 mg/kg, about 15 mg/kg, or about 20 mg/kg. In some aspects, a compound disclosed herein may be administered to a subject intravenously at least once a day, at least twice a day, at least three times a day or more. In certain aspects, a composition comprising the at least one factor as disclosed herein disclosed herein may be administered to a subject parenterally at a concentration ranging from about 0.005 mg/kg to about 20 mg/kg. In some aspects, the composition disclosed herein may be administered to a subject intravenously at a concentration of about 0.005, mg/kg, about 0.01 mg/kg, 0.05 mg/kg, about 0.1 mg/kg, about 0.5 mg/kg, about 1 mg/kg, about 2 mg/kg, about 3 mg/kg, about 4 mg/kg, about 5 mg/kg, about 10 mg/kg, about 15 mg/kg, or about 20 mg/kg. In some aspects, a composition comprising the at least one factor as disclosed herein may be administered to a subject intravenously at least once a day, at least twice a day, at least three times a day or more.

The compositions and related methods of the present disclosure, particularly administration of at least a factor to a subject, may also be used in combination with the administration of traditional therapies.

In one aspect, it is contemplated that a composition as disclosed herein is used in conjunction with additional treatment. Alternatively, the therapy may precede or follow the other agent treatment by intervals ranging from minutes to weeks. In aspects where the other agents and/or a proteins or polynucleotides are administered separately, one would generally ensure that a significant period of time did not expire between each delivery, such that the agent and the composition of the present disclosure would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations. In some exemplary aspects, the composition is administered along with umbilical cord blood transplantation after myeloablative conditioning in patients suffering from leukodystrophies. In another exemplary aspect, the composition is administered in conjunction with other drugs used for combating conditions like multiple sclerosis and variants thereof, transverse myelitis, encephalomyelitis, Guillain-Barre syndrome, progressive multifocal leukoencephalopathy, leukodystrophies, spinal cord injury, peripheral nerve damage, Parkinson's disease, amyotrophic lateral sclerosis (ALS), or Alzheimer's disease.

Various combinations may be employed, for example transplantation therapy is “A” and the factor capable of promoting myelination, is “B”: A/B/A, B/A/B, B/B/A, A/A/B, A/B/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A, A/A/B/A.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

Further, as the present inventive concept is susceptible to “aspects” or “embodiments” used interchangeably, of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [b162], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and Val to Ile or Leu.

As used herein, the terms “derivative,” “variant,” and “fragment,” when used herein with reference to a polypeptide, refers to a polypeptide related to a wild-type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild-type polypeptide.

As used herein, the term “treating” refers to the application or administration of a composition including one or more active factors to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies using a number of subjects sufficient to give a statistically significant result. As used herein, the term “ameliorate” refers to the ability to make better, or more tolerable, or reduce, a neurological, psychiatric or neuropsychatric disorder, and may encompass “limiting progression,” which refers to the lessening or limiting of the scope or severity of the neurological or psychiatric condition. The term “prevent” refers to the ability to keep a neurological, psychiatric or neuropsychatric disorder from developing, happening or existing. The term “treating” refers to the caring for, or dealing with, a neurological, psychiatric or neuropsychiatric condition either medically or surgically, and can include “ameliorating” and/or “limiting progression.” Also, within the scope of the term “treating” is the acting upon a subject with a neurological or psychiatric disorder with some factor, such as an interfering molecule, to ameliorate, improve, alter, or reduce the neurological, psychiatric or neuropsychiatric condition.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient.

The terms “comprising,” “including,” “encompassing” and “having” are used interchangeably in this disclosure. The terms “comprising,” “including,” “encompassing” and “having” mean to include, but not necessarily be limited to the things so described.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The term “administering” or “administered” as used herein is meant to include both parenteral and/or oral administration, all of which are described in more detail in the “pharmaceutical compositions” section below. By “parenteral” is meant intravenous, subcutaneous, or intramuscular administration. In the methods of the subject disclosure, the interfering molecules of the present disclosure may be administered alone, simultaneously with one or more other interfering molecule, or the compositions may be administered sequentially, in either order. It will be appreciated that the actual preferred method and order of administration will vary according to, inter alia, the particular preparation of interfering molecules being utilized, the particular formulation(s) of the one or more other interfering molecules being utilized. The optimal method and order of administration of the compositions of the disclosure for a given set of conditions can be ascertained by those skilled in the art using conventional techniques and in view of the information set out herein. The term “administering” or “administered” also refers to oral sublingual, buccal, trans nasal, transdermal, rectal, intramuscular, intravenous, intraventricular, intrathecal, and subcutaneous routes. In accordance with good clinical practice, it is preferred to administer the instant compositions at a concentration level which will produce effective beneficial effects without causing any harmful or untoward side effects.

According to the present disclosure, a “therapeutically effective amount” or “effective amount” of a pharmaceutical composition is an amount which is sufficient for the desired pharmacological effect. As used herein, “an effective amount” refers to the amount of each active factor required to confer therapeutic effect on the subject, either alone or in combination with one or more other active factors. Determination of whether an amount of the composition as disclosed herein achieved a therapeutic effect would be evident to one of skill in the art. Effective amounts vary, as recognized by those skilled in the art, depending on the condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. In some aspects, a maximum dose of the individual components or combinations thereof may be used, that is, the highest safe dose according to sound medical judgment.

### EXAMPLES

The Examples which follow are illustrative of specific aspects of the disclosure, and various uses thereof. They set forth for explanatory purposes only, and are not to be taken as limiting the disclosure.

DUOC-01 is a macrophage-like cell therapy product manufactured by culturing banked human umbilical cord blood cells under GMP conditions. Currently, the safety of DUOC-01 is being tested as a bridging therapy in children with demyelinating leukodystrophies undergoing unrelated donor umbilical cord blood transplantation after myeloablative conditioning. DUOC-01 protects against loss of function in several preclinical models with demyelinating conditions of the central nervous system, making it an attractive therapy for patients with multiple sclerosis (MS). The mechanism by which DUOC-01 promotes remyelination and if it directly influences oligodendrocyte lineage cells has been untested prior to the current disclosure. Using multiple systems: primary oligodendrocyte precursor cell (OPC) cultures, in vitro cerebellar slice cultures, and experimental autoimmune encephalomyelitis (EAE; a mouse model of MS), the mechanism by which DUOC-01 influences numerous steps of pathology and recovery were studied.

In some aspects, the current disclosure stems from extensive studies that showed that factors present in the conditioned media that was used to grow the DUOC-1 cell product can successfully convert primary OPC to myelin producing oligodendrocytes, thus promoting myelination of neurons. A mass spectrometric analysis of the conditioned media provided about 357 proteins and peptides, that may influence myelination.

### Example 1: Materials and Methods

### Manufacture of DUOC-01

The umbilical cord blood (UCB) cell suspension was washed with dextran (Hospira, Lake Forest, III.)/albumin (Grifols, Los Angeles, Calif) wash using the Sepax Cell Processing System's Cord Wash program (Biosafe), manual processing, or using the SynGenX-Lab instrument. The UCB cell suspension was then removed from the product bag and diluted in 450 mL of PBS (Life Technologies, Carlsbad, Calif) supplemented with 1% human serum albumin (HSA) and 04 μL/mL (100 units/ml benzonase nuclease (EMD Millipore, Burlington, Mass.). Cells were centrifuged and suspended in a smaller volume of PBS/HSA. Mature erythrocytes are removed using an antibody to CD235a (Glycophorin-A) and magnetic nanoparticles (EasySep™ Human Glycophorin A Depletion Kit, Stem Cell Technologies, Vancouver, Canada). The resulting cell population was suspended in Oligodendrocyte medium (α-MEM (Life Technologies, Carlsbad, Calif) supplemented with 10% fetal calf serum (Life Technologies, Carlsbad, Calif.), insulin-transferrin-selenium (Invitrogen, Carlsbad, Calif) 5 ng/mL platelet derived growth factor (PDGF) (Peprotech, Rocky Hill, N.J.), 1 ng/mL neurotrophin-3 (NT-3) (Peprotech, Rocky Hill, N.J.), 10 ng/mL vascular endothelial growth factor (VEGF) (Peprotech, Rocky Hill. N.J.), 30 ng/mL triiodothyronine (Sigma-Aldrich, St. Louis, Mo.) and plated in sterile tissue culture flasks at a concentration of 5×10⁵ cells/cm². The flasks were then incubated at 35-37° C./5% CO₂ for 21 days. On day 7 of culture, half or all of the medium was removed and replaced with an equal volume of fresh Oligodendrocyte medium. On day 14 of culture, half the volume of medium was exchanged for an equal volume of neurotrophic medium containing Neurocult NS-A basal medium (Stem Cell Technologies, Vancouver, Canada), Neurocult NS-A differentiation supplement (Stem Cell Technologies, Vancouver, Canada), and PDGF, VEGF, and NT-3 in the concentrations listed above for Oligodendrocyte medium. On day 17 of culture, half the volume of medium was exchanged for an equal volume of Oligodendrocyte medium (supplemented alpha-MEM). On day 19 of culture, one flask was harvested for initial sterility testing and characterization of the cellular content by immunophenotyping. Supplemental feeding was given if robust growth of cells was observed. On day 19-21 of culture, the remaining flasks are harvested, release and mycoplasma testing was performed, and the DUOC-01 product was formulated in its final excipient (e.g., Lactated Ringers solution) and container/closure system at the appropriate dosage for the recipient's study cohort.

### Separation of Specific Cell Populations From CB

CD14′⁺ populations from cryopreserved CB were immunomagnetically selected using Whole Blood CD14 Microbeads as described by the manufacturer (Miltenyi Biotec). Cells that did not adhere to the anti-CD14 antibody columns comprised the CD14-depleted population. Some experiments were carried out with cells from CD14V cells from freshly collected CB MNC populations depleted of erythrocytes were prepared from fresh CB either by centrifugation on Ficoll or in SepMate tubes (STEMCELL Technologies) as described by the manufacturer. CD14⁺ cells were immunomagnetically purified from MNC preparations using the CD14 Microbeads. Similar experiments were carried out with CB cell populations enriched for or depleted of CD34-expressing cells using anti-CD34 Microbeads (Milenyl Biotec).

To prepare CD14+ cell RNA for microarray analysis, freshly collected CB was centrifuged on Ficoll to prepare MNC fractions. These fractions were treated with 0.15 M NH₄Cl to lyse erythrocytes, washed in PBS, and then incubated on ice with PeCy7-mouse anti-human CD14, FITC-mouse ant-human CD3, and FITC-mouse anti-human CD235a antibodies (all from BD, San Jose, Calif.) Cells were then sorted twice by flow cytometry to yield CD14-CD235a-CD3-populations. The first enrichment sort was followed by a second purity sort. Cells were maintained at 0° C.-4° C. during all procedures, including flow sorting. The purity of selected populations and the extent of CD14+ cell depletion were determined by flow cytometry as previously described (Kurtzberg J, et al. Cytotherapy. 2015; 17 (6:803-815.)

### CPZ Demyelination in NSG Mice

Eight-week-old male NSG mice were acclimated to milled standard rodent chow for 1 week. Demyelination was subsequently induced by incorporating 0.2% by weight CPZ (bis-cyclohexanone oxaldihydrazone, Sigma-Aldrich) into the milled chow for 5 weeks. Brains were then harvested from CPZ-fed animals and controls were fed chow without CPZ for subsequent assessment of the degree of demyelination and disruption of brain histology induced by CPZ. To assess the effects of cell treatment, 2 additional groups of animals were returned to standard diet to allow remyelination. One day after the change in diet, animals were stereotactically injected in the CC (coordinates: 02 mm posterior and 1.1 mm lateral to the bregma, and 1.5 mm deep from the skull surface) with 10′ cells (DUOC-01 or CD14⁺) in 5 μl of lactated Ringer's solution or with excipient within the 2-hour expiry period for the DUOC-01 clinical cell product. One week following treatment, brains were harvested by intracardiac perfusion with PBS and then with 4% paraformaldehyde. Paraffin-embedded coronal sections were prepared for analysis of myelination status, the organization of neural fibers, and persistence of injected human cells by LFB-PAS staining, immunohistochemistry, and electron microscopy as described below. Cohorts of 5 or 6 mice were analyzed under each set of experimental conditions.

Myelination, cellular infiltration, and gliosis were assessed by LFB-PAS staining of the CC region (approximately at the level of the bregma −0.2 to −0.9 mm) (Doan V, et al. J Neurosci Res, 2013; 91(3):363-373). 5.0-μm-thick paraffin-embedded coronal sections of the CC region were used. LFB stains the myelin blue, and PAS stains demyelinated axons pink. Three independent, blinded readers scored coded LFB-PAS-stained sections between 0 and 3. A score of 3 is equivalent to the myelin status of a brain not treated with CPZ; 0 is equivalent to a completely demyelinated brain area A score of 1 or 2 corresponds to one-third or two-third fiber myelination, respectively. Similarly, a quantitative cellularity score was obtained by counting the number of nuclei in the CC region of LFB-stained brain slices on a scale of 0 to 3, by blinded readers.

### Immunohistochemistry

Brain slices from 3 animals in each treatment group were analyzed. Primary antibodies used were: rat anti-MBP (1:1,000, Abcam, Cambridge, United Kingdom); chicken anti-NFH (1:100,000, EnCor Biotech, Gainesville, Fla.) mouse anti-HuN (1:250, Millipore, Burlington, Mass.): chicken anti-GFAP (1:500, Abcam); goat anti-Iba1 (1:200, Abcam); rabbit anti-K67 (1:300, Abcam); and goat anti-Olig2 (1:50, R&D Systems, Minneapolis, Minn.). Secondary antibodies used were: Alexa-488 donkey anti-rat, Alexa-647 donkey anti-chicken, Alexa-568 donkey anti-mouse (1:500, Molecular Probes, Eugene, Oreg.). Confocal micrographs were obtained using constant settings including laser power, stack thickness, and camera resolution. The number of stained cells per microscopic field in the CC region and the average area covered by cells stained with each antibody were quantified by ImageJ software (NIH).

### Electron Microscopy

Brains were prepared for electron microscopy. Images were then analyzed using ImageJ software. For analysis, g-ratio analysis was modified such that the inner diameter of compact myelin (instead of the axon diameter) was divided by the outer diameter of the myelin sheath. Diameters were calculated from enclosed areas. Fibers with prominent outfoldings in the plane of section were excluded. A plugin for the ImageJ software (http://rsbweb.nih.gov/ij) was implemented, which allowed for semiautomated analysis of randomly selected sets of fibers (Goebbels S, et al. J Neurosc. 2010: 30(26):8953-8964). Plugin and source code are available online (http://gratio.efil.de). A minimum of 100 fibers/mouse, 3 mice/time point/treatment, were analyzed. The number of mitochondria in all cells in the CC area was counted in all the electron micrographs, and average mitochondria present per ×8,800-magnified field was calculated. To determine the size of the mitochondria, electron microscopic images were analyzed with ImageJ, using the area analysis function. For area measurement, the mitochondria were circled by the lasso tool, and then the areas of the circles were calculated and converted to their actual values using the scale bar. At least 10 images were analyzed per sample in a blinded fashion.

### Tracking DUOC-01 Cells in the Brain

DUOC-01 cells were stained with 5 μM Vybrant CFDA SE Cell Tracer dye (CFSE, V12883, green fluorescence, Life Technologies) and injected into the CC as described above. One, four, and seven days later, brains were harvested, sliced, and processed for confocal microscopy.

### Expression Analysis by Microarrays

RNA for microarray analysis was prepared from 4 flow-sorted CD14⁺ CB and 3 DUOC-01 products using the QIAGEN RNeasy Mini Kit as described by the manufacturer. These samples were used for whole-genome microarray analysis on 1 chip. Microarray analysis was performed by the Microarray Shared Resource in the Duke Center for Genomic and Computational Biology using Affymetrix GeneChip Human Transcriptome Array 2.0 microarrays. Partek (Genomics Suite 6.6 (Partek Inc.) was used to perform data analysis. Robust multichip analysis (RMA) normalization was performed on the entire dataset. Multi-way ANOVA and analysis of the fold change were performed to select target genes that were differentially expressed. Hierarchical clustering was performed on differentially expressed genes based on average linkage with Pearson's dissimilarity.

### Statistics

In most cases statistical comparisons were conducted with 2-tailed Student's t tests with unequal variance. For comparing LFB and cellularity scores, Wilcoxon rank-sun tests were used. Statistical comparisons were performed using the Wilcoxon rank-sum test for clustered data using the clusrank package in R. Mean differences were considered significant if P values were less than 0.05.

### Example 2: Characterization of DUOC-01 Cell Product

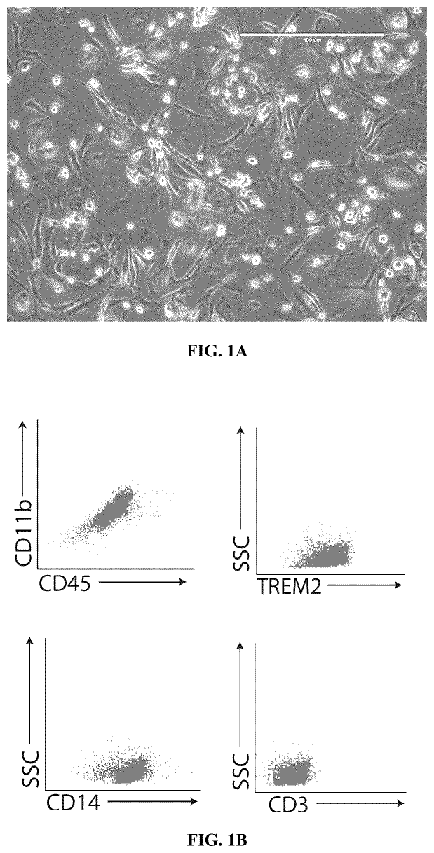

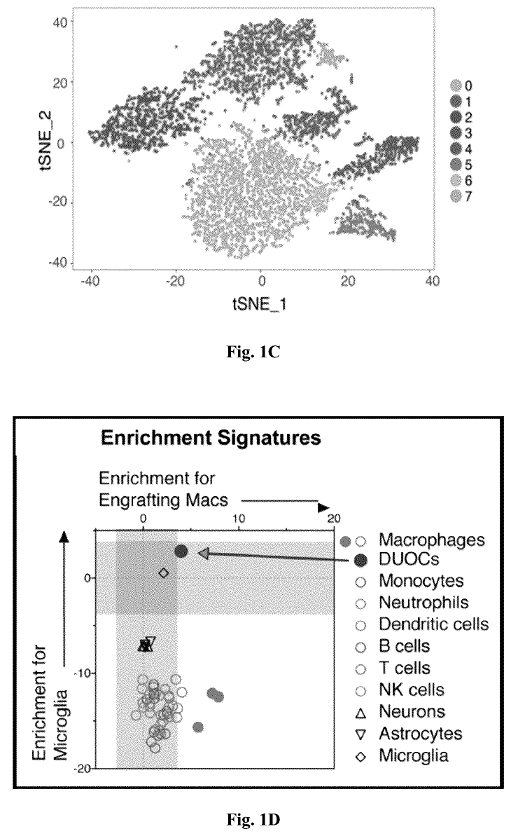

DUOC-01 cell product was established essentially as provided in Example 1. FIG. 1A shows an image of the DUOC-01 cell population. The cell population was further characterized using FACS and RNA seq for expression of various markers including CD3, CD45, CD11b, CD14, CD16, CD206, CD163, Iba1, HLA-DR, TREM 2, and iNOS macrophage or microglia markers, as provided in FIG. 1B. These studies showed that DUOC-01 cell population is enriched in cells expression of CD11b, CD45, TREM2, CD14, while being depleted of SSC, and CD3. Flow cytometric analysis showed that positively selected populations contained greater than 90% CD14⁺ cells and depleted populations contained less than 2% CD14⁺ cells.

To summarize, DUOC-01 cell product was shown to be a heterogenous population of macrophage-like cells as seen in FIG. 1C. Additionally, it was found that compared to peripheral macrophages, DUOC-01 cells are enriched for microglial signatures FIG. 1D. Gene expression studies showed that several genes that promote oligodendrocyte maturation are upregulated in the DUOC-01 cell product. Bioplex analysis further showed that DUOC-01 cells secrete a range of factors that promote remyelination.

### Example 3: DUOC-01 Cells Show Remyelination Potential in an Organotypic Cerebellar Culture Model of LPC Mediated Demyelination

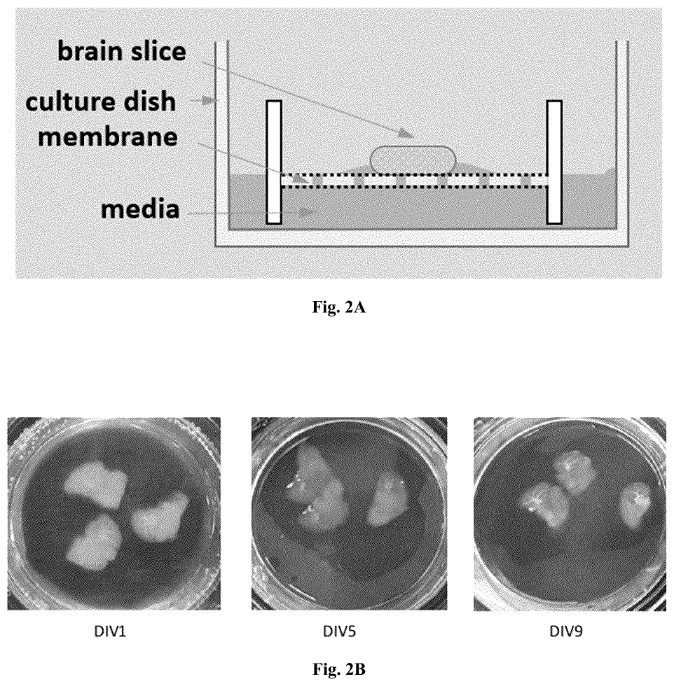

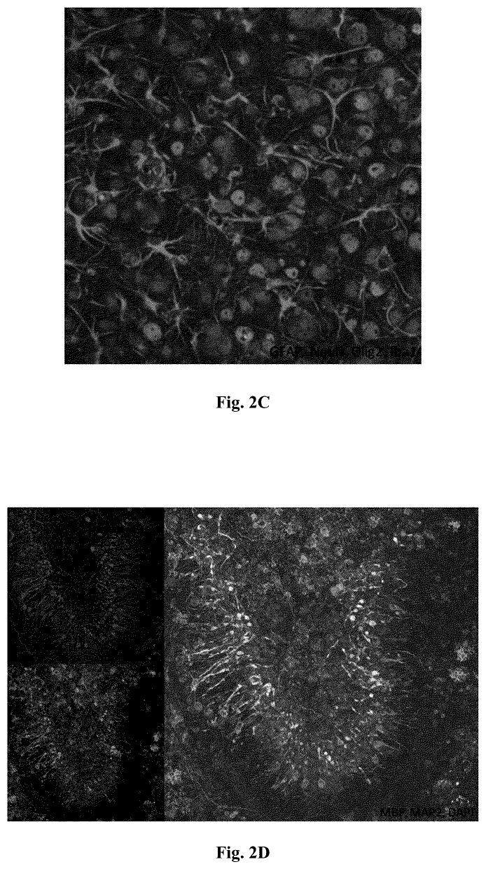

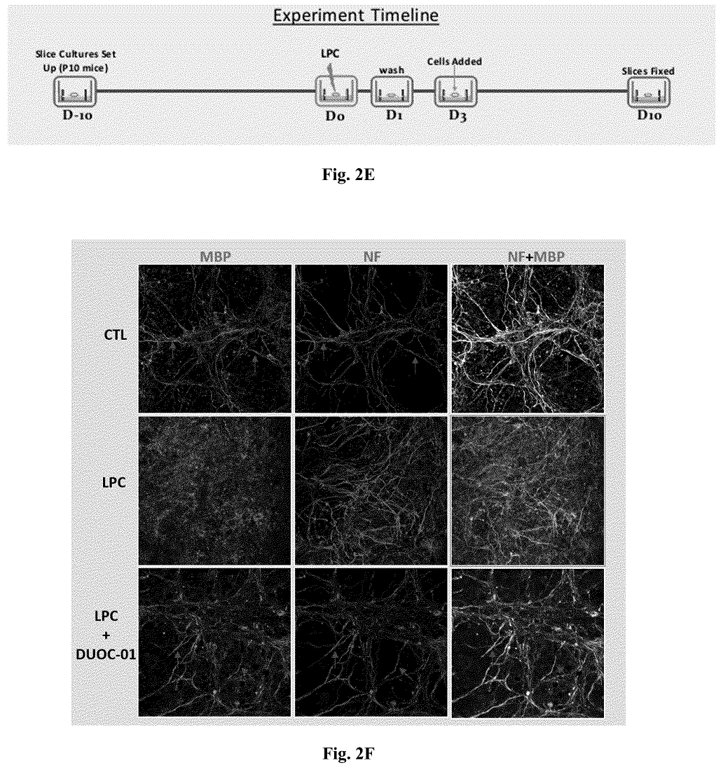

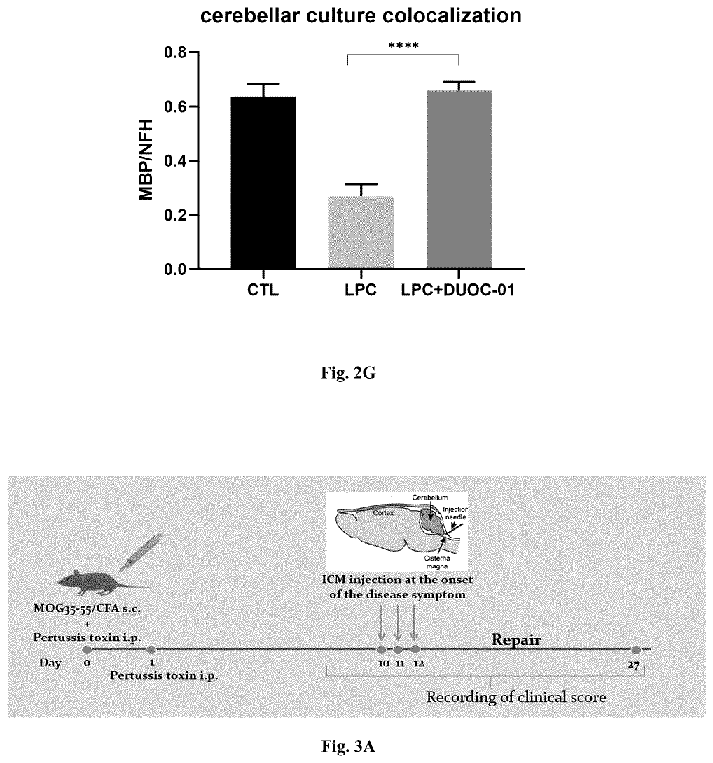

An ex vivo cerebellar slice culture was set up essentially as shown in FIG. 2A. A 0.4 μm membrane insert was used to hold the tissue slice in a six-well plate. The cultures were treated with lysophosphatidylcholine (LPC) to induce demyelination. The culture was photographed at various stages (see FIG. 2B: days in vitro (DIV)). Immunohistochemical analysis was done on brain slices to check the presence and morphology of cell types within the mouse fore-brain slices in ex-vivo culture conditions. A representative image as shown in FIG. 2C with astrocytes, neurons, oligodendrocytes and microglial cells. Immunohistochemical staining was also conducted to check the presence of myelinated axons in the cerebellar slices in ex-vivo culture conditions (FIG. 2D).

To check if DUOC-01 can support remyelination, slice cultures were set up and treated with LPC as shown previously. DUOC-01 cells were added to the culture on day 3 and slices fixed for staining at day 10 (see schematic in FIG. 2E). Results show that DUOC-01 cells are capable for inducing remyelination potential in culture model. Immunohistochemical staining for myelin basic protein (MBP) and neurofilaments (NFH) was conducted after LPC and DUOC-0 treatment to check for remyelination (FIG. 2F). Remyelination levels were estimated based on co-localization of MBP/NFH as shown in FIG. 2G. A higher number of myelinated neuron fibers were present in the DUOC-treated group compared to LPC group, demonstrating a clear enhancement in myelination in the presence of DUOC-01.

### Example 4: DUOC-01 Cells Demonstrate Therapeutic Effect in Murine Model of Experimental Auto-Immune Encephalomyelitis

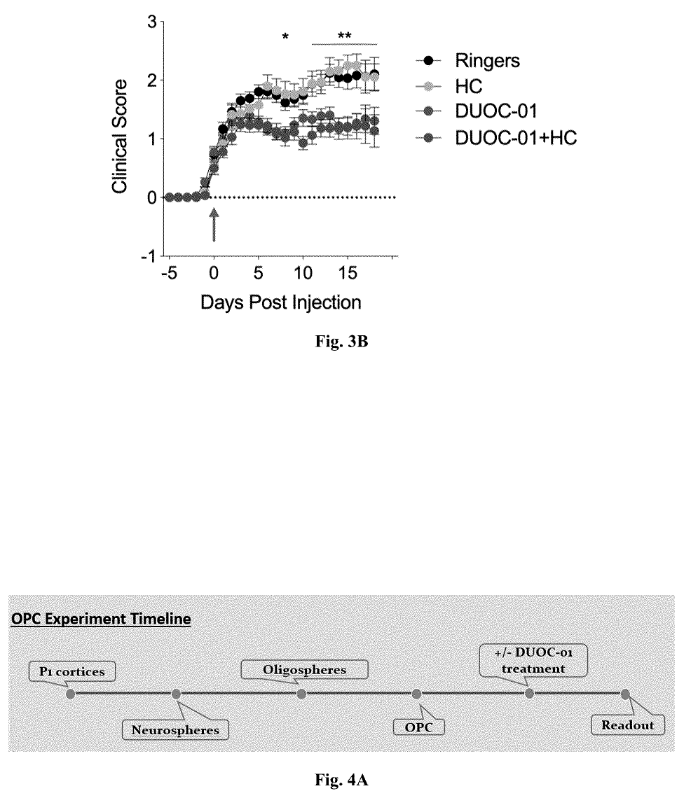

To test the DUOC-01 effect in the EAE model, C57BL/6 mice were immunized with myelin oligodendrocyte glycoprotein peptide (MOG35-55) in complete Freund's adjuvant. To match clinical protocols, DUOC-01 was incubated in Ringer's Lactate with hydrocortisone (HC) for 2 hours at room temperature. At the onset of EAE disease symptoms, DUOC-01 were injected into the cerebrospinal fluid by a single intra-cisterna magna injection. Clinical scores were recorded daily for two weeks (see schematic in FIG. 3A). Compared to mice injected with Ringer's or HC+Ringer's, mice injected with DUOC-01 derived clinical benefits with lower clinical scores (FIG. 3B).

### Example 5: DUOC-01 Drives the Maturation of the OPC Culture

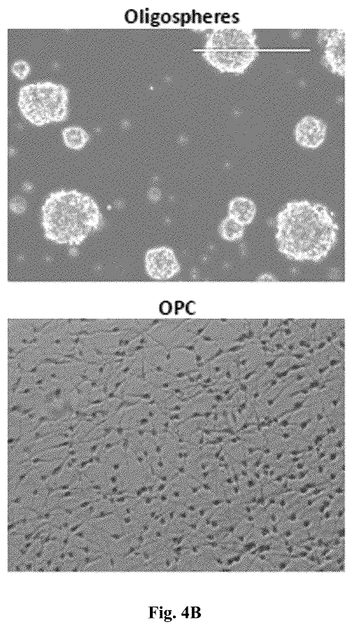

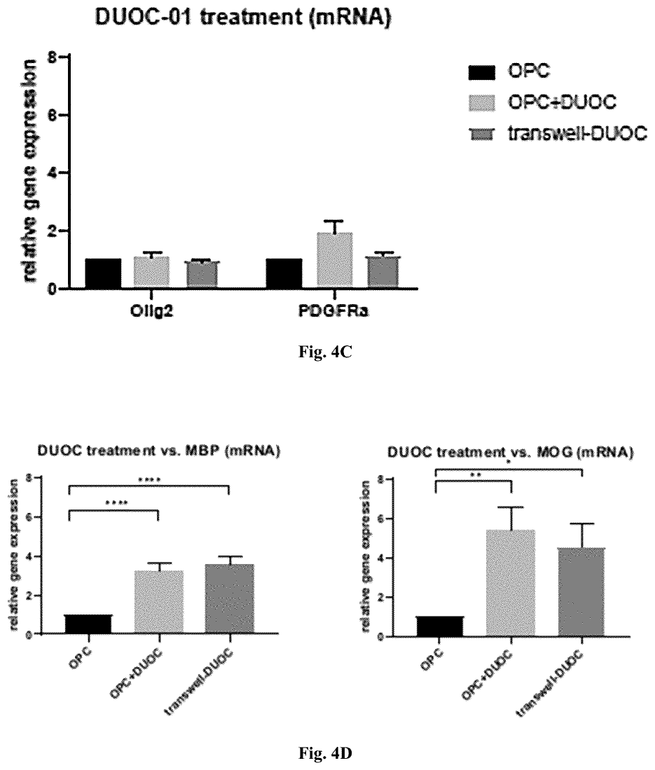

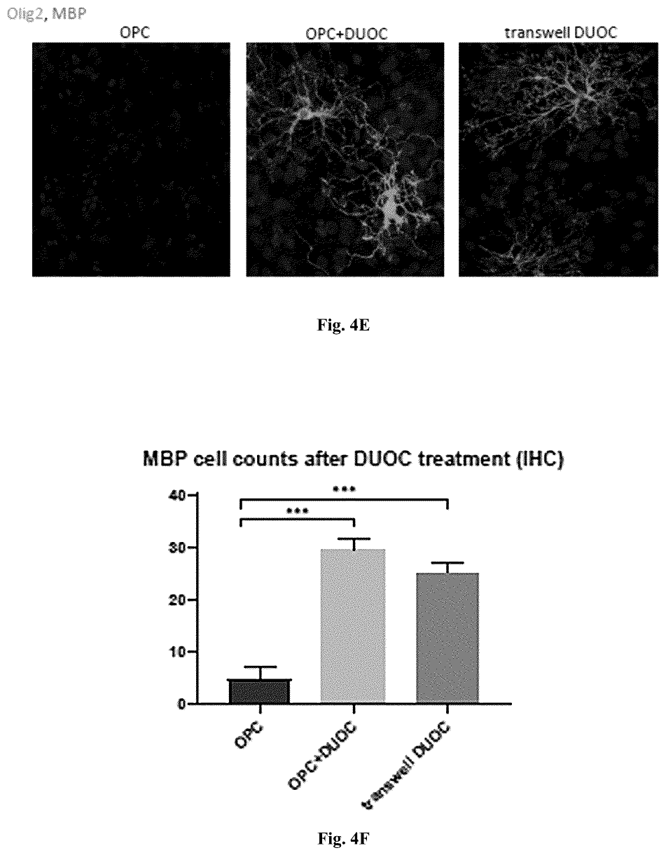

To test if DUOC-01 could directly affect OPCs, a primary OPC culture isolated from neonatal mice was set-up. A timeline of the experimental approach is provided in FIG. 4A. Formation of oligospheres and morphology of OPC is shown in FIG. 4B. DUOC-01 were added to the cultures during the differentiation phase. DUOC-01 treatment did not affect the OPC proliferation whether the DUOC-01 were added directly to the OPC culture or in the transwell (see FIG. 4C). Addition of DUOC-01 however skewed the culture to a more mature phenotype. Mature oligodendrocyte markers Myelin Basic Protein (MBP) and Myelin Oligodendrocyte Glycoprotein (MOG), both were upregulated after treatment (FIG. 4D). This was further confirmed with immunohistochemistry with Olig2 and MBP staining (FIG. 4E). DUOC-01 decreased the amount of Ki67⁺/Oligo2⁺ OPCs and increased the amount of GalC⁺ oligodendrocytes. Quantitation of the IHC for MBP staining showed that significantly higher MBP expression could be seen after direct DUOC-01 treatment of the treatment from DUOC-01 in the transwell (FIG. 4F). These data indicate that DUOC-01 drive the maturation of OPC to become myelin producing oligodendrocytes. Overall, this data suggest that DUOC-01 could be beneficial in treating MS and other diverse neurological demyelinating conditions. Additionally factors from DUOC-01 as present in the transwell, are fully capable of replicating the beneficial effects of DUOC-01 when added directly.

### Example 6: Remy-Macs from DUOC-01 Cultures can Promote Maturation of OPCs

The ability of Remy-Macs (clarified conditioned media) derived from conditioned media obtained from culturing DUOC-01 cells to promote the differentiation of OPCs to mature myelin producing oligodendrocytes was tested. For this, primary mouse OPC cultures were established as provided below.

Briefly, neurospheres were first obtained. Cerebrum from P0-P1 C57BL/6 pups was mechanically dissociated by pipetting with a 1 ml pipette. The cell suspension was filtered through a 70 μM cell strainer and plated in a 25 cm² culture flask in the DMEM/F12/B27 media containing 10 ng/ml EGF, which was called P0. Floating neurospheres were passaged at 1:2 ratio in the same media every five days. To drive to oligospheres, passage 2-6 neurospheres were dissociated by pipetting 10 times and re-suspended in DMEM/F12/B27 containing 10 ng/ml PDGFaa and 50 ng/ml bFGF. The floating cell aggregates were passaged at 1:2 ratio every five days. P2-P6 oligospheres were used to set up OPC culture. To set up OPC culture, the floating oligospheres were collected and dissociated with 1 ml 10× TryLE for 15 minutes at 37° C. water bath. After pipetting 10 times with fire polished glass pipette, the cells were digested for another 5 minutes at 37° C. The digestion was stopped by diluting with 40 ml of DMEM/F12/B27 media. After filtered through a 40 μM cell strainer, the single cell suspension was counted and plated at the Poly-D-Lysin coated 24-well plate at 1×10⁵/well for 4-5 days before treating with Remy-Macs. OPC cultures were either cultured in OPC media (control; CTL) or with Remy-Macs (conditioned media taken from macrophages derived from umbilical cord blood monocytes).

DUOC-01 culture set-up: DUOC-01 cultures were set up essentially as provided, with cryopreserved cord blood units thawed and washed to obtain white blood cells to culture in Oligo media for 21 days to grow into DUOC-01.

To obtain Remy-Macs, at D20 of the DUOC-01 culture, media was removed and replaced with 10 ml of DMEM/F12/B27. Remy-Macs were collected the next day after the removal of the debris in the supernatant by spinning at 300×g for 10 min.

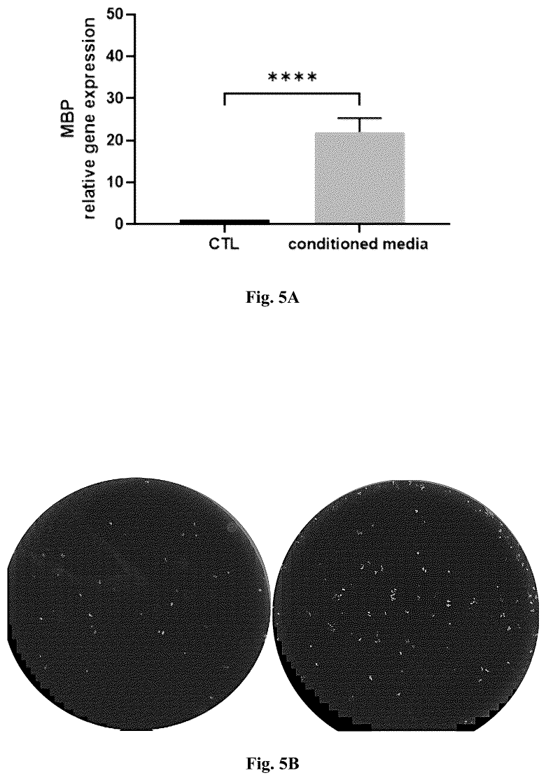

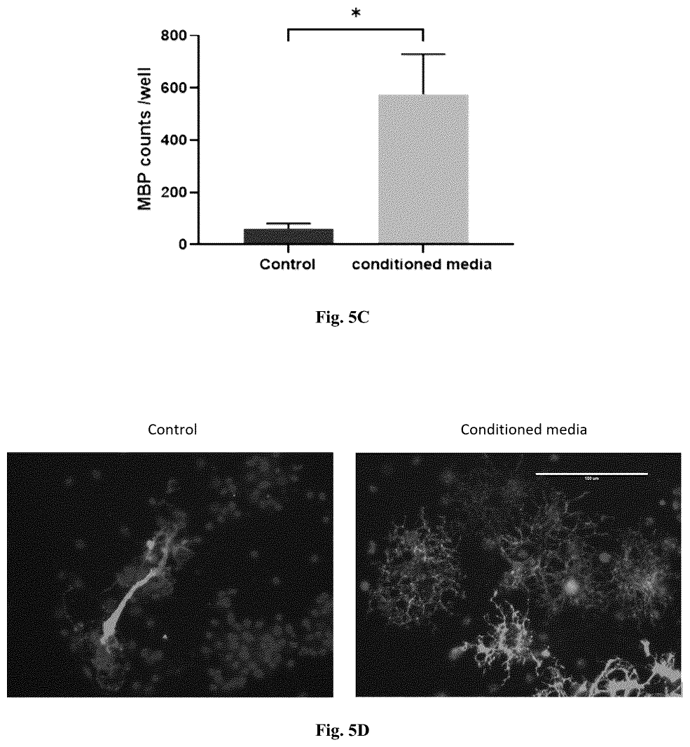

To test if Remy-Macs promote the maturation of OPCs, OPC cultures were either cultured in OPC media (control; CTL) or with Remy-Macs (conditioned media taken from macrophages derived from umbilical cord blood monocytes). OPC cultures were exposed to control media (DMEM/F12/B27) or Remy-Macs (see above). After 5-7 days, the media was removed, and cells were harvested to extract RNA. Quantitative real-time PCR was performed to measure the MBP relative gene expression after the cDNA synthesis. Myelin basic protein (MBP) is a marker for mature oligodendrocytes. Therefore, MBP mRNA was analyzed by quantitative real-time PCR. Results show that Remy-Macs increased the expression of MBP compared to CTL in a statistically significant manner (see FIG. 5A, N=27, t-test, ****P<0.0001).

This was further confirmed by whole well tile scans of OPCs cultures treated with control media or Remy-Macs (see FIGS. 5B and 5C, N=4, t-test, *P<0.05). OPCs were immunostained using an anti-MBP antibody to detect the MBP expression. The tile scan images were taken using an Evos microscope to show MBP expressing cells over the entire culture. The number of MBP expressing cells were counted using ImageJ.

Immunohistochemistry was performed using anti-MBP antibody for mature oligodendrocytes and cells were co-labeled with DAPI to stain for nuclei. The 40× images were taken using an Evos system. Representative images of OPC cultures treated with control media or Remy-Macs and immune labeled for MBP are provided in FIG. 5D. In cultures treated with Remy-Macs, cells displayed complex highly ramified processes demonstrating the maturation of OPC into mature oligodendrocytes. In cultures treated with control media, cells displayed simple morphology demonstrating immature OPCs.

### Example 7: Mass Spectrometric Analysis of Remy-Macs

Remy-Macs from 5 different cell DUOC-01 cultures and conditioned medium from suitable control were cleaned using acetone and re-dissolved into 200 uL ABC/SDC solution prior to analyzing by nanoLC-MS/MS (N=5). Proteins were identified and 357 were determined to be enhanced in by five Remy-Macs tested (see Table 1).

TABLE 1
Proteins identified by Mass Spectrometric Analysis

| # | Protein Name |
|---|---|
| 1 | 1,4-alpha-glucan-branching enzyme |
| 2 | 10 kDa heat shock protein, mitochondrial |
| 3 | 14-3-3 protein epsilon |
| 4 | 14-3-3 protein gamma |
| 5 | 14-3-3 protein theta |
| 6 | 14-3-3 protein zeta/delta |
| 7 | 3-ketoacyl-CoA thiolase, mitochondrial |
| 8 | 45 kDa calcium-binding protein |
| 9 | 60 kDa heat shock protein, mitochondrial |
| 10 | 60S ribosomal protein L10A |
| 11 | 6-phosphogluconate dehydrogenase, decarboxylating |
| 12 | 6-phosphogluconolactonase |
| 13 | 72 kDa type IV collagenase |
| 14 | 78 kDa glucose-regulated protein |
| 15 | Acid ceramidase |
| 16 | Actin-related protein 2 |
| 17 | Actin-related protein 2/3 complex subunit 1B |
| 18 | Actin-related protein 2/3 complex subunit 2 |
| 19 | Actin-related protein 2/3 complex subunit 3 |
| 20 | Actin-related protein 2/3 complex subunit 4 |
| 21 | Actin-related protein 2/3 complex subunit 5 |
| 22 | Actin-related protein 3 |
| 23 | Acyl-CoA-binding protein |
| 24 | ADAM DEC1 |
| 25 | Adenosine deaminase 2 |
| 26 | Adenosylhomocysteinase |
| 27 | Adenylyl cyclase-associated protein 1 |
| 28 | Alcohol dehydrogenase [NADP(+)] |
| 29 | Aldose 1-epimerase |
| 30 | Aldose reductase |
| 31 | Allograft inflammatory factor 1 |
| 32 | Alpha-1-antitrypsin |
| 33 | Alpha-2-HS-glycoprotein |
| 34 | Alpha-2-macroglobulin |
| 35 | Alpha-actinin-1 |
| 36 | Alpha-actinin-4 |
| 37 | Alpha-enolase |
| 38 | Alpha-fetoprotein |
| 39 | Alpha-galactosidase A |
| 40 | Aminopeptidase B |
| 41 | Annexin A1 |
| 42 | Annexin A2 |
| 43 | Annexin A5 |
| 44 | Antithrombin-III |
| 45 | Apolipoprotein E |
| 46 | Aspartate aminotransferase, cytoplasmic |
| 47 | Aspartate aminotransferase, mitochondrial |
| 48 | Astrocytic phosphoprotein PEA-15 |
| 49 | Basigin |
| 50 | Beta-2-microglobulin |
| 51 | Beta-galactosidase |
| 52 | Beta-hexosaminidase subunit alpha |
| 53 | Beta-hexosaminidase subunit beta |
| 54 | Biliverdin reductase A |
| 55 | Brain acid soluble protein 1 |
| 56 | BTB/POZ domain-containing protein KCTD12 |
| 57 | Calmodulin |
| 58 | Calreticulin |
| 59 | Calumenin |
| 60 | Carbonic anhydrase 2 |
| 61 | Carbonyl reductase [NADPH] 1 |
| 62 | Carboxypeptidase Q |
| 63 | Cartilage oligomeric matrix protein |
| 64 | Catalase |
| 65 | Cathepsin B |
| 66 | Cathepsin D |
| 67 | Cathepsin L1 |
| 68 | Cathepsin S |
| 69 | Cathepsin Z |
| 70 | Cation-independent mannose-6-phosphate receptor |
| 71 | CD109 antigen |
| 72 | CD166 antigen |
| 73 | CD44 antigen |
| 74 | CD59 glycoprotein |
| 75 | Cellular retinoic acid-binding protein 2 |
| 76 | Chitinase-3-like protein 1 |
| 77 | Chitotriosidase-1 |
| 78 | Chloride intracellular channel protein 1 |
| 79 | Citrate synthase, mitochondrial |
| 80 | Clathrin heavy chain 1 |
| 81 | Coactosin-like protein |
| 82 | Cofilin-1 |
| 83 | Collagen alpha-1(I) chain |
| 84 | Collagen alpha-1(III) chain |
| 85 | Collagen alpha-1(VI) chain |
| 86 | Complement C1q subcomponent subunit A |
| 87 | Complement C1q subcomponent subunit B |
| 88 | Complement C1q subcomponent subunit C |
| 89 | complement C2 |
| 90 | Complement C3 |
| 91 | Complement C4-A |
| 92 | Complement factor D |
| 93 | Copper transport protein ATOX1 |
| 94 | Coronin-1B |
| 95 | Coronin-1C |
| 96 | Costars family protein ABRACL |
| 97 | C-type mannose receptor 2 |
| 98 | C-X-C motif chemokine 5 |
| 99 | Cystatin-B |
| 100 | Cystatin-C |
| 101 | Cytochrome c |
| 102 | Cytoplasmic aconitate hydratase |
| 103 | Cytosolic non-specific dipeptidase |
| 104 | D-dopachrome decarboxylase |
| 105 | Deoxyribonuclease-2-alpha |
| 106 | Destrin |
| 107 | Dihydrolipoyl dehydrogenase, mitochondrial |
| 108 | Dihydropyrimidinase-related protein 2 |
| 109 | Dipeptidyl peptidase 1 |
| 110 | Dipeptidyl peptidase 2 |
| 111 | Dipeptidyl peptidase 3 |
| 112 | Disintegrin and metalloproteinase domain-containing protein 9 |
| 113 | Elongation factor 1-alpha 1 |
| 114 | Elongation factor 2 |
| 115 | Endoplasmic reticulum resident protein 29 |
| 116 | Endoplasmin |
| 117 | Endothelial protein C receptor |
| 118 | Epididymal secretory protein E1 |
| 119 | Eukaryotic initiation factor 4A-I |
| 120 | Extracellular matrix protein 1 |
| 121 | Ezrin |
| 122 | F-actin-capping protein subunit alpha-1 |
| 123 | F-actin-capping protein subunit alpha-2 |
| 124 | Fascin |
| 125 | Fatty acid-binding protein, adipocyte |
| 126 | Fatty acid-binding protein, epidermal |
| 127 | Fatty acid-binding protein, heart |
| 128 | Fermitin family homolog 3 |
| 129 | Ferritin light chain |
| 130 | Fibrinogen beta chain |
| 131 | Fibulin-1 |
| 132 | Flavin reductase (NADPH) |
| 133 | Fructose-1,6-bisphosphatase 1 |
| 134 | Fructose-bisphosphate aldolase A |
| 135 | Fumarylacetoacetase |
| 136 | Galectin-1 |
| 137 | Galectin-3 |
| 138 | Gamma-interferon-inducible lysosomal thiol reductase |
| 139 | Ganglioside GM2 activator |
| 140 | Glia maturation factor beta |
| 141 | Glia maturation factor gamma |
| 142 | Glucose-6-phosphate 1-dehydrogenase |
| 143 | Glucose-6-phosphate isomerase |
| 144 | Glucosidase 2 subunit beta |
| 145 | Glutamate dehydrogenase 1, mitochondrial |
| 146 | Glutamate--cysteine ligase catalytic subunit |
| 147 | Glutaredoxin-1 |
| 148 | Glutathione reductase, mitochondrial |
| 149 | Glutathione S-transferase omega-1 |
| 150 | Glutathione S-transferase P |
| 151 | Glutathione synthetase |
| 152 | Glyceraldehyde-3-phosphate dehydrogenase |
| 153 | Glycogen phosphorylase, liver form |
| 154 | Glyoxalase domain-containing protein 4 |
| 155 | Granulins |
| 156 | Growth factor receptor-bound protein 2 |
| 157 | guanine nucleotide-binding protein G(i) subunit alpha-2 |
| 158 | heat shock 70 kDa protein 1A |
| 159 | Heat shock 70 kDa protein 4 |
| 160 | Heat shock cognate 71 kDa protein |
| 161 | Heat shock protein HSP 90-alpha |
| 162 | Heat shock protein HSP 90-beta |
| 163 | Hemoglobin subunit alpha |
| 164 | Hemopexin |
| 165 | Hepatocyte growtH factor activator |
| 166 | Hexokinase-3 |
| 167 | Histone H4 |
| 168 | Hydroxyacylglutathione hydrolase, mitochondrial |
| 169 | Immunoglobulin heavy constant gamma 1 |
| 170 | Importin subunit beta-1 |
| 171 | Insulin-like growth factor-binding protein 4 |
| 172 | Integrin alpha-X |
| 173 | Integrin beta-2 |
| 174 | Inter-alpha-trypsin inhibitor heavy chain H2 |
| 175 | Inter-alpha-trypsin inhibitor heavy chain H3 |
| 176 | Isocitrate dehydrogenase [NADP] cytoplasmic |
| 177 | Isoform 2 of F-actin-capping protein subunit beta |
| 178 | Isoform 2 of Triosephosphate isomerase |
| 179 | Isoform 2 of Tropomyosin alpha-3 chain |
| 180 | Isoform Non-brain of Clathrin light chain A |
| 181 | Kynureninase |
| 182 | Lactotransferrin |
| 183 | Legumain |
| 184 | Leukocyte elastase inhibitor |
| 185 | Leukotriene A-4 hydrolase |
| 186 | LIM and SH3 domain protein 1 |
| 187 | Lipoprotein lipase |
| 188 | L-lactate dehydrogenase A chain |
| 189 | L-lactate dehydrogenase B chain |
| 190 | Low affinity immunoglobulin gamma Fc region receptor III-A |
| 191 | Lumican |
| 192 | Lymphocyte-specific protein 1 |
| 193 | lysosomal alpha-glucosidase |
| 194 | Lysosomal alpha-mannosidase |
| 195 | lysosomal protective protein |
| 196 | Lysosome-associated membrane glycoprotein 1 |
| 197 | Lysosome-associated membrane glycoprotein 2 |
| 198 | lysozyme c |
| 199 | Macrophage colony-stimulating factor 1 |
| 200 | Macrophage colony-stimulating factor 1 receptor |
| 201 | macrophage mannose receptor 1 |
| 202 | Macrophage Migration inhibitory factor |
| 203 | Macrophage-capping protein |
| 204 | Malate dehydrogenase, cytoplasmic |
| 205 | Malate dehydrogenase, mitochondrial |
| 206 | Matrilysin |
| 207 | Matrix metalloproteinase-9 |
| 208 | Metalloproteinase inhibitor 1 |
| 209 | Metalloproteinase inhibitor 2 |
| 210 | Moesin |
| 211 | Monocyte differentiation antigen CD14 |
| 212 | Myosin-9 |
| 213 | Myotrophin |
| 214 | Myristoylated alanine-rich C-kinase substrate |
| 215 | N-acetyl-D-glucosamine kinase |
| 216 | N-acetylglucosamine-6-sulfatase |
| 217 | NADP-dependent malic enzyme |
| 218 | Neuroblast differentiation-associated protein AHNAK |
| 219 | Neuropilin-1 |
| 220 | Neuropilin-2 |
| 221 | Neutral alpha-glucosidase AB |
| 222 | Nicotinamide phosphoribosyltransferase |
| 223 | Nucleobindin-1 |
| 224 | Nucleolin |
| 225 | Peptidyl-prolyl cis-trans isomerase A |
| 226 | Peptidyl-prolyl cis-trans isomerase B |
| 227 | Peptidyl-prolyl cis-trans isomerase FKBP1A |
| 228 | Peroxiredoxin-1 |
| 229 | Peroxiredoxin-2 |
| 230 | Peroxiredoxin-6 |
| 231 | Phosphatidylethanolamine-binding protein 1 |
| 232 | Phosphoenolpyruvate carboxykinase [GTP], mitochondrial |
| 233 | Phosphoglucomutase-2 |
| 234 | Phosphoglycerate kinase 1 |
| 235 | Phosphoglycerate mutase 1 |
| 236 | Phospholipase D3 |
| 237 | Phospholipid transfer protein |
| 238 | Pigment epithelium-derived factor |
| 239 | Plasminogen |
| 240 | Plastin-2 |
| 241 | Platelet basic protein |
| 242 | Platelet-activating factor acetylhydrolase |
| 243 | Pleckstrin homology domain-containing family O member 2 |
| 244 | Prelamin-A/C |
| 245 | Probable serine carboxypeptidase CPVL |
| 246 | Procollagen-lysine, 2-oxoglutarate 5-dioxygenase 1 |
| 247 | Profilin-1 |
| 248 | Programmed cell death 6-interacting protein |
| 249 | Pro-low-density lipoprotein receptor-related protein 1 |
| 250 | Prosaposin |
| 251 | Prostaglandin reductase 1 |
| 252 | Prostaglandin-H2 D-isomerase |
| 253 | Proteasome activator complex subunit 1 |
| 254 | Proteasome activator complex subunit 2 |
| 255 | Proteasome subunit alpha type-1 |
| 256 | Proteasome subunit alpha type-2 |
| 257 | Proteasome subunit alpha type-3 |
| 258 | Proteasome subunit alpha type-4 |
| 259 | Proteasome subunit alpha type-5 |
| 260 | Proteasome subunit alpha type-6 |
| 261 | Proteasome subunit alpha type-7 |
| 262 | Proteasome subunit beta type-1 |
| 263 | Proteasome subunit beta type-8 |
| 264 | Proteasome subunit beta type-9 |
| 265 | Protein disulfide-isomerase |
| 266 | Protein disulfide-isomerase A3 |
| 267 | Protein disulfide-isomerase A4 |
| 268 | Protein disulfide-isomerase A6 |
| 269 | Protein FAM49B |
| 270 | Protein S100-A11 |
| 271 | Protein S100-A4 |
| 272 | Protein/nucleic acid deglycase DJ-1 |
| 273 | Protein-glutamine gamma-glutamyltransferase 2 |
| 274 | Protein-L-isoaspartate(D-aspartate) O-methyltransferase |
| 275 | Prothrombin |
| 276 | Purine nucleoside phosphorylase |
| 277 | Puromycin-sensitive aminopeptidase |
| 278 | Pyridoxal kinase |
| 279 | Pyruvate kinase PKM |
| 280 | Quinone oxidoreductase PIG3 |
| 281 | Rab GDP dissociation inhibitor alpha |
| 282 | Rab GDP dissociation inhibitor beta |
| 283 | Radixin |
| 284 | Ras GTPase-activating-like protein IQGAP1 |
| 285 | Ras-related protein Rap-1b |
| 286 | Renin receptor |
| 287 | Reticulon-4 |
| 288 | Retinal dehydrogenase 1 |
| 289 | Retinol-binding protein 4 |
| 290 | Rho GDP-dissociation inhibitor 1 |
| 291 | Rho GDP-dissociation inhibitor 2 |
| 292 | Ribonuclease inhibitor |
| 293 | Ribonuclease pancreatic |
| 294 | Ribonuclease T2 |
| 295 | Ribosyldihydronicotinamide dehydrogenase [quinone] |
| 296 | S-adenosylmethionine synthase isoform type-2 |
| 297 | Scavenger receptor cysteine-rich type 1 protein M130 |
| 298 | Serglycin |
| 299 | Serotransferrin |
| 300 | Serpin B6 |
| 301 | Serum albumin |
| 302 | SH3 domain-binding glutamic acid-rich-like protein |
| 303 | SH3 domain-binding glutamic acid-rich-like protein 3 |
| 304 | SLAM family member 5 |
| 305 | Sorbitol dehydrogenase |
| 306 | Sparc |
| 307 | Stabilin-1 |
| 308 | Stress-induced-phosphoprotein 1 |
| 309 | Sulfhydryl oxidase 1 |
| 310 | Superoxide dismutase [Cu—Zn] |
| 311 | Superoxide dismutase [Mn], mitochondrial |
| 312 | Synaptic vesicle membrane protein VAT-1 homolog |
| 313 | Talin-1 |
| 314 | Tartrate-resistant acid phosphatase type 5 |
| 315 | Tetranectin |
| 316 | thioredoxin |
| 317 | Thioredoxin domain-containing protein 17 |
| 318 | Thioredoxin-dependent peroxide reductase, mitochondrial |
| 319 | Thrombospondin-1 |
| 320 | Thymidine phosphorylase |
| 321 | Thymosin beta-4 |
| 322 | Tissue alpha-L-fucosidase |
| 323 | Transaldolase |
| 324 | Transcobalamin-2 |
| 325 | Transferrin receptor protein 1 |
| 326 | Transforming growth factor-beta-induced protein ig-h3 |
| 327 | Transgelin-2 |
| 328 | Transitional endoplasmic reticulum ATPase |
| 329 | Transketolase |
| 330 | Translationally-controlled tumor protein |
| 331 | Transmembrane glycoprotein NMB |
| 332 | Tripeptidyl-peptidase 1 |
| 333 | Tropomyosin alpha-4 chain |
| 334 | Tryptophan--tRNA ligase, cytoplasmic |
| 335 | Tubulin beta chain |
| 336 | Tubulin-specific chaperone A |
| 337 | Twinfilin-2 |
| 338 | Tyrosine-protein phosphatase non-receptor type 6 |
| 339 | Tyrosine-protein phosphatase non-receptor type substrate 1 |
| 340 | Ubiquitin carboxyl-terminal hydrolase isozyme L1 |
| 341 | Ubiquitin thioesterase otub1 |
| 342 | Ubiquitin-conjugating enzyme E2 L3 |
| 343 | Ubiquitin-conjugating enzyme E2 variant 1 |
| 344 | UMP-CMP kinase |
| 345 | Urokinase plasminogen activator surface receptor |
| 346 | UTP--glucose-1-phosphate uridylyltransferase |
| 347 | UV excision repair protein RAD23 homolog B |
| 348 | Vasodilator-stimulated phosphoprotein |
| 349 | Vimentin |
| 350 | Vinculin |
| 351 | Vitamin D-binding protein |
| 352 | V-type proton ATPase catalytic subunit A |
| 353 | V-type proton ATPase subunit B, brain isoform |
| 354 | V-type proton ATPase subunit S1 |
| 355 | WD repeat-containing protein 1 |
| 356 | Xaa-Pro dipeptidase |
| 357 | Zyxin |

The polypeptides with enhanced levels in the RemyMacs were further investigated to identify a list of probable candidate factors that support maturation of oligodendrocytes. The bioactive compounds of interest were narrowed down by cross-referencing databases with ligand-receptor pairs and further narrowed by receptor expression on oligodendrocyte precursor cells.

Based on these studies, the following factors were identified for further studies: TGM2 Transglutaminase-2 (TGM2), Apolipoprotein E (APOE), Calreticulin (CALR), Collagen Type I Alpha 1 Chain (COL1A1), Alpha-2-Macroglobulin (A2M), Complement C1q B Chain (C1QB), Heat Shock Protein 90 Beta Family Member 1 (HSP90B1), Heat Shock Protein 90 Alpha Family Class A Member 1 (HSP90AA1), Lactotransferrin (LTF), Matrix Metallopeptidase 9 (MMP9), Thrombospondin 1 (THBS1), Vinculin (VCL), Plasminogen (PLG), or Serpin Family C Member 1 (SERPINC1).

1. A composition comprising at least one factor present in an umbilical cord blood derived macrophage cell population culture medium or a biologically active variant, derivative or fragment thereof, and at least one excipient, wherein the composition promotes myelination of a neuron in presence of a primary oligodendrocyte precursor cell (OPCs).

2. The composition of claim 1, comprising at least two, or at least 5, or at least 10, or at least 50, or at least 100, or at least 150, or at least 200, or at least 300 or more factors present in an umbilical cord blood derived macrophage cell population, or biologically active variants, derivatives, or fragments thereof.

3. The composition of claim 2, wherein the composition comprises Remy-Macs.

4. The composition of claim 2, wherein the factor is one or more of the factors listed in Table 1.

5. The composition of claim 4, wherein the factor is one or more of Transglutaminase-2 (TGM2), Apolipoprotein E (APOE), Calreticulin (CALR), Collagen Type I Alpha 1 Chain (COL1A1), Alpha-2-Macroglobulin (A2M), Complement C1q B Chain (C1QB), Heat Shock Protein 90 Beta Family Member 1 (HSP90B1), Heat Shock Protein 90 Alpha Family Class A Member 1 (HSP90AA1), Lactotransferrin (LTF), Matrix Metallopeptidase 9 (MMP9), Thrombospondin 1 (THBS1), Vinculin (VCL), Plasminogen (PLG), or Serpin Family C Member 1 (SERPINC1) or any combination of thereof.

6. The composition of claim 1, wherein administering an effective amount of the composition in a subject in need thereof can reduce, reverse, or treat a demyelination condition.

7. The composition of claim 1, wherein the umbilical cord blood derived macrophage cell population comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6 and IL-10.

8. The composition of claim 7, wherein the umbilical cord blood derived macrophage cell population comprises a DUOC-01 cell product.

9. The composition of claim 6, wherein the demyelination condition is multiple sclerosis, leukodystrophies, spinal cord injury, peripheral nerve damage, Parkinson's disease, amyotrophic lateral sclerosis (ALS), or Alzheimer's disease.

10. The composition of claim 9, wherein the subject is a child suffering from leukodystrophies and undergoing donor umbilical cord blood transplantation after myeloablative conditioning.

11. The composition of claim 6, wherein the administering is done via a subcutaneous, intrathecal, intramuscular, intranasal, transepidermal, parenteral, transepithelial, or epidural route.

12. The composition of claim 1, wherein the at least one factor can drive the differentiation of the OPC to mature myelin basic protein expressing oligodendrocytes.

13. The composition of claim 12, wherein the OPCs are present in an in vitro or ex vivo culture, or are present or administered in vivo.

14. A method of promoting myelination of neurons, the method comprising:
a. obtaining at least one factor present in an umbilical cord blood derived macrophage cell population culture medium, or a biologically active variant, derivative or fragment thereof; and
b. contacting at least a primary oligodendrocyte precursor (OPC) cell with the at least one factor, wherein the OPC matures to a myelin basic protein expressing oligodendrocyte, thus promoting myelination is neurons.

15. The method of claim 14, wherein the umbilical cord blood derived macrophage cell population comprises cells expressing one or more of CD45, CD11b, CD14, CD16, CD206, CD163, Iba1, HLA-DR, TREM 2, and iNOS macrophage or microglia markers; and wherein such cells secrete IL-6 and IL-10.

16. The method of 15, wherein step b is performed in vitro, ex vivo or in vivo by administering to a subject in need thereof the at least one factor present in the umbilical cord blood derived macrophage cell population.

17. The method of claim 16, wherein the administering is done via a subcutaneous, intrathecal, intramuscular, intranasal, parenteral, transepithelial, or epidural route.

18. The method of claim 16, wherein the subject has symptoms of or is diagnosed with multiple sclerosis, leukodystrophies, spinal cord injury, peripheral nerve damage, Parkinson's disease, amyotrophic lateral sclerosis (ALS), or Alzheimer's disease.

19. A method of treating a subject in need thereof, the method comprising:
a. determining an amount of at least one factor secreted by an umbilical cord blood derived macrophage cell population, in a biological sample of the subject in need thereof;
b. comparing the amount of the at least one factor with a standard to obtain a differential amount;
c. determining a treatment regimen for the subject, wherein the treatment regimen comprises administering to the subject in need thereof a therapeutically effective amount of a composition comprising the at least one factor, or a biologically active variant, derivative or fragment thereof.

20. The method of claim 20, wherein the subject is exhibiting symptoms of or is diagnosed with multiple sclerosis, leukodystrophies, spinal cord injury, peripheral nerve damage, Parkinson's disease, amyotrophic lateral sclerosis (ALS), or Alzheimer's disease.
